(19) United States
(12) Patent Application Publication
(10) Pub. No.: US 2007/0292401 A1
(43) Pub. Date: Dec. 20, 2007

(54) Soft tissue repair and regeneration using stem cell products

(76) Inventors: Alexander M. Harmon, Clinton, NJ (US); Laura J. Brown, Hamilton Square, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/471,215

(22) Filed: Jun. 20, 2006

(51) Int. Cl. A61K 48/00; C12N 5/08; C12N 5/06

(52) U.S. Cl. 424/93.21; 435/325; 435/366

(57) ABSTRACT

Stem cells products having the potential to support cells of a soft tissue lineage, and methods of preparation and use of those stem cell products are disclosed. The invention also relates to methods for the use of such stem cells products in the regeneration and repair of soft tissue, and in cell-based therapies for of soft tissue conditions.

### FIELD OF THE INVENTION

This invention relates to the field of mammalian cell biology and cell culture, in particular, the invention relates to stem cells products having the potential to support cells of a soft tissue lineage, and methods of preparation and use of those stem cell products. The invention also relates to methods for the use of such stem cells products in the regeneration and repair of soft tissue, and in cell-based therapies for of soft tissue conditions.

### BACKGROUND OF THE INVENTION

Soft tissue conditions, including medical conditions, such as injury to soft tissue are quite common. Injuries to soft tissue, include for example, vascular, skin, or musculoskeletal tissue, are quite common. One example of a fairly common soft tissue injury is damage to the pelvic floor. This is a potentially serious medical condition that may occur during childbirth or from complications thereof which can lead to damage to the vesicovaginal fascia. Such an injury can result in a cystocele, which is a herniation of the bladder. Similar medical conditions include rectoceles (a herniation of the rectum), enteroceles (a protrusion of the intestine through the rectovaginal or vesicovaginal pouch), and enterocystoceles (a double hernia in which both the bladder and intestine protrude).

Another common soft tissue injury is a hernia. The basic manifestation of a hernia is a protrusion of an organ into a defect within the fascia. Surgical approaches toward hernia repair have focused on reducing the presence of the hernial contents in the peritoneal cavity and generating a firm closure of the fascial defect either by using prosthetic, allogeneic, or autologous materials. A number of techniques have been used to produce this closure including the movement of autologous tissues and the use of synthetic mesh products. Drawbacks to these current products and procedures include hernia recurrence upon weakening of the closure.

As another example of a soft tissue condition, ligaments and tendons are viscoelastic structures that mediate normal joint movement and stability and are subject to tear and brittleness with age or injury. These structures are complex, relatively static collagenous structures with functional links to the bone, muscle, menisci, and other nearby tendons and ligaments.

Soft tissue conditions further include, for example, conditions of skin (e.g., ischemic wounds, diabetic wounds, scar revision or the treatment of traumatic wounds, severe burns, skin ulcers (e.g., decubitus (pressure) ulcers, venous ulcers, and diabetic ulcers), and surgical wounds such as those associated with the excision of skin cancers); vascular conditions (e.g., vascular disease such as peripheral arterial disease, abdominal aortic aneurysm, carotid disease, and venous disease; vascular injury; and improper vascular development); conditions affecting vocal cords; cosmetic conditions (e.g., those involving repair, augmentation, or beautification); muscle diseases (e.g., congenital myopathies; myasthenia gravis; inflammatory, neurogenic, and myogenic muscle diseases; and muscular dystrophies such as Duchenne muscular dystrophy, Becker muscular dystrophy, myotonic dystrophy, limb-girdle-muscular dystrophy, facioscapulohumeral muscular dystrophy, congenital muscular dystrophies, oculopharyngeal muscular dystrophy, distal muscular dystrophy, and Emery-Dreifuss muscular dystrophy); conditions of connective tissues such as tendons and ligaments, including but not limited to a periodontal ligament and anterior cruciate ligament; and conditions of organs and/or fascia (e.g., the bladder, intestine, pelvic floor).

Surgical approaches to correct soft tissue conditions or defects in the body generally involve the implantation of structures made of biocompatible, inert materials that attempt to replace or substitute for the defective function. Implantation of non-biodegradable materials results in permanent structures that remain in the body as a foreign object. Implants that are made of resorbable materials are suggested for use as temporary replacements where the object is to allow the healing process to replace the resorbed material. However, these approaches have met with limited success for the long-term correction of structures in the body.

Thus, novel therapeutic regimens for treatment of soft tissue conditions are of great clinical significance.

### SUMMARY OF THE INVENTION

The invention is generally directed to stem cell products (SCPs) including cell fractions such as, soluble cell fractions; insoluble cell fractions; cell lysates, supernates of cell fractions; cell membrane-containing fractions, and combinations thereof having the potential to provide support to a cell, for example, a soft tissue cell phenotype. The term stem cell products (SCPs) is more fully defined and described herein.

In some embodiments, the stem cell products are derived from an embryonic source of cells including, but not limited to embryonic cells obtained from the embryoid bodies including blastocysts, trophoblasts, the inner cells mass, as well as embryonic germ cells. Also the stem cells may be obtained from postpartum tissues including, but not limited to placenta, umbilical cord, amnioic epithelium, amnionic membrane, and cells obtained from amnionic fluid. Also the stem cells may be obtained from adult stem cells incuding, but not limited to mesenchymal stem cells derived from bone marrow and mesenchymal like stem cells including, but not limited to adipose derived stem cells, epidermal derived stem cells, hair follicle derived stem cells, mammary tissue derived stem cells, olfactory derived stem cells, neural stem cells, epithelial stem cell, cardiac derived stem cells, and stem cells derived from teeth. Also the stem cells may be hematopoietic stem cells including, but not limited to umbilical cord blood derived hematopoietic stem cells.

In some embodiments the invention provides compositions of one or more SCP and one or more bioactive factors, including, but not limited to growth factors, anti-apoptotic agents, anti-inflammatory agents, and/or differentiation-inducing factors. Some compositions of the invention comprise one or more SCP and one or more other cell types, for example, epithelial cells (e.g., cells of oral mucosa, gastrointestinal tract, nasal epithelium, respiratory tract epithelium, vaginal epithelium, and corneal epithelium), bone marrow cells, adipocytes, stem cells, keratinocytes, melanocytes, dermal fibroblasts, vascular endothelial cells (e.g., aortic endothelial cells, coronary artery endothelial cells, pulmonary artery endothelial cells, iliac artery endothelial cells, microvascular endothelial cells, umbilical artery endothelial cells, umbilical vein endothelial cells, and endothelial progenitors (e.g., CD34+, CD34+/CD117+ cells)), myoblasts, myocytes, stromal cells, and other soft tissue cells or progenitor cells.

Some embodiments of the invention provide a matrix combined with one or more SCPs for administration to a patient. The SCP may be substantially homogeneous or heterogeneous. For example, the matrix may be inoculated with SCP and cells of at least one other desired cell type, including, but not limited to epithelial cells (e.g., cells of oral mucosa, gastrointestinal tract, nasal epithelium, respiratory tract epithelium, vaginal epithelium, and corneal epithelium), bone marrow cells, adipocytes, stem cells, keratinocytes, melanocytes, dermal fibroblasts, vascular endothelial cells (e.g., aortic endothelial cells, coronary artery endothelial cells, pulmonary artery endothelial cells, iliac artery endothelial cells, microvascular endothelial cells, umbilical artery endothelial cells, umbilical vein endothelial cells, and endothelial progenitors (e.g., CD34+, CD34+/CD117+ cells)), myoblasts, myocytes, stromal cells, and other soft tissue cells or progenitor cells. The matrix may contain or be pre-treated with one or more bioactive factors including, for example, drugs, anti-inflammatory agents, antiapoptotic agents, and growth factors. The seeded or pre-treated matrices can be introduced into a patient's body in any way known in the art including, but not limited to implantation, injection, surgical attachment, transplantation with other tissue, and the like. The matrices of the invention may be configured in vitro or in vivo to a desired shape and/or size, for example, to the shape and/or size of a tissue or organ in vivo. The matrix may be in the form of a tissue engineering scaffold. The scaffolds of the invention may be flat or tubular or may comprise sections thereof. The scaffolds of the invention may be multilayered. Matrices of the invention may comprise or be pre-treated with any one or more of the foregoing SCP-products.

In some embodiments, SCPs provide trophic support to a soft tissue cell. Examples of soft tissue cells offered trophic support by SCPs include cells of cartilage tissue, meniscal tissue, ligament tissue, tendon tissue, intervertebral disc tissue, periodontal tissue, skin tissue, vascular tissue, muscle tissue, fascia tissue, periosteal tissue, ocular tissue, pericardial tissue, lung tissue, synovial tissue, nerve tissue, kidney tissue, bone marrow, urogenital tissue, intestinal tissue, liver tissue, pancreas tissue, spleen tissue, or adipose tissue.

In some embodiments, pharmaceutical compositions of SCPs are provided. The pharmaceutical compositions preferably include a pharmaceutically acceptable carrier or excipient.

In some embodiments, methods of regenerating soft tissue in a patient by administering SCPs, SCP compositions, or SCP matrices of the invention to a patient are provided.

In some embodiments, methods for treating a soft tissue condition in a patient by administering one or more SCPs are provided. Treatment of a soft tissue condition includes but is not limited to trophic support of soft tissue, tissue repair, tissue reconstruction, tissue bulking, cosmetic treatment, therapeutic treatment, tissue augmentation, and tissue sealing. The SCPs may be used in the treatment of, for example but not by way of limitation, a hernia, damage to the pelvic floor, a burn, cancer, traumatic injury, scars, skin ulcers (e.g., decubitus (pressure) ulcers, venous ulcers, and diabetic ulcers), ischemic wounds, surgical wounds such as those associated with the excision of skin cancers; vascular disease such as peripheral arterial disease, abdominal aortic aneurysm, carotid disease, and venous disease; muscle disease (e.g., congenital myopathies; myasthenia gravis; inflammatory, neurogenic, and myogenic muscle diseases; and muscular dystrophies such as Duchenne muscular dystrophy, Becker muscular dystrophy, myotonic dystrophy, limb-girdle-muscular dystrophy, facioscapulohumeral muscular dystrophy, congenital muscular dystrophies, oculopharyngeal muscular dystrophy, distal muscular dystrophy, and Emery-Dreifuss muscular dystrophy); and replacement and repair of connective tissues such as tendons and ligaments (e.g., anterior cruciate ligament, rotator cuff, periodontal ligament).

The invention further provides methods of providing trophic support to cells such as soft tissue cells by exposing or contacting a cell to one or more SCPs. Examples of soft tissue cells for which SCPs may provide trophic support include a stem cell, a myocyte, a myoblast, a keratinocyte, a melanocyte, a dermal fibroblast, a bone marrow cell, an adipocyte, an epithelial cell, a stromal cell, and an endothelial cell (e.g., aortic endothelial cells, coronary artery endothelial cells, pulmonary artery endothelial cells, iliac artery endothelial cells, microvascular endothelial cells, umbilical artery endothelial cells, umbilical vein endothelial cells, and endothelial progenitors (e.g., CD34+, CD34+/CD117+ cells). Such exposure of the soft tissue cell may stimulate angiogenesis. Methods of the invention further include methods of inducing angiogenesis by exposing a soft tissue cell to a SCP product. Examples of soft tissue cells that form endothelial networks in accordance with the methods of the invention include aortic endothelial cells, coronary artery endothelial cells, pulmonary artery endothelial cells, iliac artery endothelial cells, microvascular endothelial cells, umbilical artery endothelial cells, umbilical vein endothelial cells, and endothelial progenitors (e.g., CD34+, CD34+/CD117+ cells). Methods of providing trophic support or stimulating angiogenesis of the invention may be effected in vitro or in vivo.

Methods of the invention also include methods of treating a patient in need of angiogenic factors by administering to a patient one or more SCPs.

Also provided by the invention are methods of producing a vascular network. In some embodiments, the methods of producing a vascular network involve exposing or contacting a population of soft tissue cells to one or more SCPs. The population of soft tissue cells preferably contains at least one soft tissue cell of an aortic endothelial cell, coronary artery endothelial cell, pulmonary artery endothelial cell, iliac artery endothelial cell, microvascular endothelial cell, umbilical artery endothelial cell, and umbilical vein endothelial cell. The method of producing a vascular network may be performed in vitro or in vivo. The invention also encompasses the vascular networks produced by the methods of the invention. Methods of treating a condition such as a soft tissue condition in a patient by administering the vascular networks also are provided. In some embodiments, the soft tissue condition is a vascular condition, such as a vascular disease or injury or improper vascular development. In some embodiments, the vascular network is administered by transplantation to the patient.

Further provided by the invention are kits of the SCPs. The kits preferably include at least one component of a matrix, a hydrating agent, a cell culture substrate, a bioactive factor, a second cell type, a differentiation-inducing agent, cell culture media, and instructions, for example, for culturing cells or for administration of the cell products.

Other features and advantages of the invention will be apparent from the detailed description and examples that follow.

### DETAILED DESCRIPTION OF ILLUSTRATIVE EMBODIMENTS

### Definitions

Various terms used throughout the specification and claims are defined as set forth below.

The term stem cell products is defined to mean cellular components or cell products having the potential to provide support to a cell. Cellular components include, but are not limited to soluble cell fractions; insoluble cell fractions; cell membrane-containing fractions; cell cytoplasm-containing fractions; cell nucleus-containing fractions; cell lysates; supernates of cell fractions; conditioned medium; extracellular matrix; components of any of the foregoing and combinations thereof. Cell products include, but are not limited to trophic and other biological factors produced naturally by SCPs or through genetic modification, or through conditioned medium from SPC culture. The terms stem cell products, SCP and SCPs are used interchangeably herein.

Stem cells are undifferentiated cells defined by their ability at the single cell level to both self-renew and differentiate to produce progeny cells, including self-renewing progenitors, non-renewing progenitors and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

Stem cells are classified by their developmental potential as: (1) totipotent—able to give rise to all embryonic and extraembryonic cell types; (2) pluripotent—able to give rise to all embryonic cell types; (3) multipotent—able to give rise to a subset of cell lineages, but all within a particular tissue, organ, or physiological system (for example, hematopoietic stem cells (HSC) can produce progeny that include HSC (self-renewal), blood cell-restricted oligopotent progenitors, and all cell types and elements (e.g., platelets) that are normal components of the blood); (4) oligopotent—able to give rise to a more restricted subset of cell lineages than multipotent stem cells; and (5) unipotent—able to give rise to a single cell lineage (e.g., spermatogenic stem cells).

Stem cells are also categorized on the basis of the source from which they may be obtained. An adult stem cell is generally a multipotent undifferentiated cell found in tissue comprising multiple differentiated cell types. The adult stem cell can renew itself and, under normal circumstances, differentiate to yield the specialized cell types of the tissue from which it originated, and possibly other tissue types. An embryonic stem cell is a pluripotent cell from the inner cell mass of a blastocyst-stage embryo. A fetal stem cell is one that originates from fetal tissues or membranes. A postpartum stem cell is a multipotent or pluripotent cell that originates substantially from extraembryonic tissue available after birth, namely, the placenta and the umbilicus. These cells have been found to possess features characteristic of pluripotent stem cells, including rapid proliferation and the potential for differentiation into many cell lineages. Postpartum stem cells may be blood-derived (e.g., as are those obtained from umbilical cord blood) or non-blood-derived (e.g., as obtained from the non-blood tissues of the umbilical cord and placenta).

Embryonic tissue is typically defined as tissue originating from the embryo (which in humans refers to the period from fertilization to about six weeks of development. Fetal tissue refers to tissue originating from the fetus, which in humans refers to the period from about six weeks of development to parturition. Extraembryonic tissue is tissue associated with, but not originating from, the embryo or fetus. Extraembryonic tissues include extraembryonic membranes (chorion, amnion, yolk sac and allantois), umbilical cord and placenta (which itself forms from the chorion and the maternal decidua basalis).

Differentiation is the process by which an unspecialized (“uncommitted”) or less specialized cell acquires the features of a specialized cell, such as a nerve cell or a muscle cell, for example. A differentiated or differentiation-induced cell is one that has taken on a more specialized (“committed”) position within the lineage of a cell. The term committed, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

In a broad sense, a progenitor cell is a cell that has the capacity to create progeny that are more differentiated than itself and yet retains the capacity to replenish the pool of progenitors. By that definition, stem cells themselves are also progenitor cells, as are the more immediate precursors to terminally differentiated cells. When referring to the cells as described in greater detail below, this broad definition of progenitor cell may be used. In a narrower sense, a progenitor cell is often defined as a cell that is intermediate in the differentiation pathway, i.e., it arises from a stem cell and is intermediate in the production of a mature cell type or subset of cell types. This type of progenitor cell is generally not able to self-renew. Accordingly, if this type of cell is referred to herein, it will be referred to as a non-renewing progenitor cell or as an intermediate progenitor or precursor cell.

Various terms are used to describe cells in culture. Cell culture refers generally to cells taken from a living organism and grown under controlled conditions (“in culture”). A primary cell culture is a culture of cells, tissues or organs taken directly from organisms and before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

A cell line is a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to the seeding density, substrate, medium, and time between passaging.

Generally, a trophic factor is defined as a substance that promotes survival, growth, proliferation, maturation, differentiation, and/or maintenance of a cell, or stimulates increased activity of a cell. Trophic support is used herein to refer to the ability to promote survival, growth, proliferation, maturation, differentiation, and/or maintenance of a cell, or to stimulate increased activity of a cell.

When referring to cultured vertebrate cells, the term senescence (also replicative senescence or cellular senescence) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are actually resistant to programmed cell death (apoptosis), and have been maintained in their nondividing state for as long as three years. These cells are very much alive and metabolically active, but they do not divide. The nondividing state of senescent cells has not yet been found to be reversible by any biological, chemical, or viral agent.

The term isolated refers to a cell, cellular component, or a molecule that has been removed from its native environment.

The term about refers to an approximation of a stated value within a range of ±10%.

Soft tissue, as used herein, refers generally to extraskeletal structures found throughout the body and includes, but is not limited to cartilage tissue, meniscal tissue, ligament tissue, tendon tissue, intervertebral disc tissue, periodontal tissue, skin tissue, vascular tissue, muscle tissue, fascia tissue, periosteal tissue, ocular tissue, pericardial tissue, lung tissue, synovial tissue, nerve tissue, kidney tissue, bone marrow, urogenital tissue, intestinal tissue, liver tissue, pancreas tissue, spleen tissue, or adipose tissue, and combinations thereof.

Soft tissue condition (or injury or disease) is an inclusive term encompassing acute and chronic conditions, disorders or diseases of soft tissue, and medical conditions. For example, the term encompasses conditions caused by disease or trauma or failure of the tissue to develop normally. Examples of soft tissue conditions include, but are not limited to hernias, damage to the pelvic floor, tear or rupture of a tendon or ligament, skin wounds (e.g., scars, traumatic wounds, ischemic wounds, diabetic wounds, severe burns, skin ulcers (e.g., decubitus (pressure) ulcers, venous ulcers, and diabetic ulcers), and surgical wounds such as those associated with the excision of skin cancers); cosmetic conditions (e.g. reconstructive surgery and tissue bulking); vascular conditions (e.g., vascular disease such as peripheral arterial disease, abdominal aortic aneurysm, carotid disease, and venous disease; vascular injury, improper vascular development); muscle diseases (e.g., congenital myopathies; myasthenia gravis; inflammatory, neurogenic, and myogenic muscle diseases; and muscular dystrophies such as Duchenne muscular dystrophy, Becker muscular dystrophy, myotonic dystrophy, limb-girdle-muscular dystrophy, facioscapulohumeral muscular dystrophy, congenital muscular dystrophies, oculopharyngeal muscular dystrophy, distal muscular dystrophy, and Emery-Dreifuss muscular dystrophy).

The term treating (or treatment of) a soft tissue condition refers to ameliorating the effects of, or delaying, halting or reversing the progress of, or delaying or preventing the onset of, a soft tissue condition as defined herein and includes trophic support of soft tissue, soft tissue repair, reconstruction (e.g., breast reconstruction), bulking, cosmetic treatment, therapeutic treatment, tissue augmentation (e.g., bladder augmentation), and tissue sealing.

The term effective amount refers to a concentration of a reagent or pharmaceutical composition, such as a growth factor, differentiation agent, trophic factor, cell population or other agent, that is effective for producing an intended result, including cell growth and/or differentiation in vitro or in vivo, or treatment of a soft tissue condition as described herein. With respect to growth factors, an effective amount may range from about 1 nanogram/milliliter to about 1 microgram/milliliter. With respect to SCP as administered to a patient in vivo, an effective amount may range from from about 1 nanogram/milliliter to about 1 microgram/milliliter or from about 1 nanogram/centimeter squared of implantation site to about 1 milligram/centimeter squared of implanataion of implantation site. It will be appreciated that the mass of SCP to be administered will vary depending on the specifics of the condition to be treated, including but not limited to size or total volume/surface area to be treated, as well as proximity of the site of administration to the location of the region to be treated, among other factors familiar to the medicinal biologist.

The terms effective period (or time) and effective conditions refer to a period of time or other controllable conditions (e.g., temperature, humidity for in vitro methods), necessary or preferred for an agent or pharmaceutical composition to achieve its intended result.

The term patient or subject refers to animals, including mammals, preferably humans, who are treated with the pharmaceutical compositions or in accordance with the methods described herein.

The term pharmaceutically acceptable carrier (or medium), which may be used interchangeably with the term biologically compatible carrier or medium, refers to reagents, cells, compounds, materials (including, for example, matrices), compositions, and/or dosage forms which are, within the scope of sound medical judgment, suitable for use in contact with the tissues of human beings and animals without excessive toxicity, irritation, allergic response, or other complication commensurate with a reasonable benefit/risk ratio. As described in greater detail herein, pharmaceutically acceptable carriers suitable for use in the present invention include liquids, semi-solid (e.g., gels) and solid materials (e.g., scaffolds). As used herein, the term biodegradable describes the ability of a material to be broken down (e.g., degraded, eroded, dissolved) in vivo. The term biodegradable includes degradation in vivo with or without elimination (e.g., by resorption) from the body. The semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways. Examples include, but are not limited to, hyaluronic acid and saline.

The term matrix as used herein refers to a support for the SCP, for example, a scaffold (e.g., wonen or nonwoven fiberous scaffold, foams such as PCL/PGA, or self-assembling peptides such as RAD16) or other supporting medium (e.g., hydrogel or a biomaterial such as Collagen/oxidized regenerated cellulose).

The following abbreviations are used herein:

ANG2 (or Ang2) for angiopoietin 2;

APC for antigen-presenting cells;

BDNF for brain-derived neurotrophic factor;

bFGF for basic fibroblast growth factor;

bid (BID) for “bis in die” (twice per day);

BSP for bone sialoprotein;

CK18 for cytokeratin 18;

CXC ligand 3 for chemokine receptor ligand 3;

DAPI for 4′-6-Diamidino-2-phenylindole-2HCl;

DMEM for Dulbecco's Modified (or Minimal) Essential Medium;

DMEM:lg (or DMEM:Lg, DMEM:LG) for DMEM with low glucose;

EDTA for ethylene diamine tetraacetic acid;

EGF (or E) for epidermal growth factor;

EPO for erythropoietin;

FACS for fluorescent activated cell sorting;

FBS for fetal bovine serum;

FGF (or F) for fibroblast growth factor;

GCP-2 for granulocyte chemotactic protein-2;

GDF-5 for growth and differentiation factor 5;

GFAP for glial fibrillary acidic protein;

HB-EGF for heparin-binding epidermal growth factor;

HCAEC for Human coronary artery endothelial cells;

HGF for hepatocyte growth factor;

hMSC for Human mesenchymal stem cells;

HNF-1alpha for hepatocyte-specific transcription factor;

HUVEC for Human umbilical vein endothelial cells;

I309 for a chemokine and the ligand for the CCR8 receptor and is responsible for chemoattraction of TH2 type T-cells;

IGF for insulin-like growth factor;

IL-6 for interleukin-6;

IL-8 for interleukin 8;

K19 for keratin 19;

K8 for keratin 8;

KGF for keratinocyte growth factor;

MCP-1 for monocyte chemotactic protein 1;

MDC for macrophage-derived chemokine;

MIP1alpha for macrophage inflammatory protein 1alpha;

MIP1beta for macrophage inflammatory protein 1beta;

MMP for matrix metalloprotease (MMP);

MSC for mesenchymal stem cells;

NHDF for Normal Human Dermal Fibroblasts;

NPE for Neural Progenitor Expansion media;

OxLDLR for oxidized low density lipoprotein receptor;

PBMC for peripheral blood mononuclear cell;

PBS for phosphate buffered saline;

PDC for placenta-derived cell;

PDGFbb for platelet derived growth factor;

PDGFr-alpha for platelet derived growth factor receptor alpha;

PD-L2 for programmed—death ligand 2;

PE for phycoerythrin;

PO for “per os” (by mouth);

SCP for postpartum-derived cell;

Rantes (or RANTES) for regulated on activation, normal T cell expressed and secreted;

rb for rabbit;

rh for recombinant human;

SC for subcutaneously;

SCID for severe combined immunodeficiency;

SDF-1alpha for stromal-derived factor 1alpha;

SHH for sonic hedgehog;

SMA for smooth muscle actin;

SOP for standard operating procedure;

TARC for thymus and activation-regulated chemokine;

TCP for tissue culture plastic;

TGFbeta2 for transforming growth factor beta2;

TGFbeta-3 for transforming growth factor beta-3;

TIMP1 for tissue inhibitor of matrix metalloproteinase 1;

TPO for thrombopoietin;

TuJ1 for BIII Tubulin;

hUTC for umbilicus-derived cell;

VEGF for vascular endothelial growth factor;

vWF for von Willebrand factor; and

alphaFP for alpha-fetoprotein.

### Stem Cell Source

Various patents and other publications are cited herein and throughout the specification, each of which is incorporated by reference herein in their entirety.

In one embodiment, the invention provides stem cell products (SCPs) including cell fractions (e.g., soluble cell fractions; insoluble cell fractions; cell lysate, supernates of cell fractions; cell membrane-containing fractions). SCPs are derived from stem cells. The stem cells may be of an embryonic source including, but not limited to embryonic cells obtained from the embryoid bodies including blastocysts, trophoblasts, the inner cells mass, as well as embryonic germ cells. Also the stem cells may be obtained from postpartum tissues including, but not limited to placenta, umbilical cord, amnionic epithelium, amnionic membrane, and cells obtained amnionic fluid. Also the stem cells may be obtained from adult stem cells including, but to limited to mesenchymal stem cells derived from bone marrow and mesenchymal like stem cells including, but not limited to adipose derived stem cells, epidermal derived stem cells, hair follicle derived stem cells, mammary tissue derived stem cells, olfactory derived stem cells, neural stem cells, epithelial stem cell, cardiac derived stem cells, and stem cells derived from teeth. Also the stem cells may be hematopoietic stem cells including but not limited to umbilical cord blood derived hematopoietic stem cells.

SCPs can be derived from any type of stem cell including, but not limited to cells of mesodermal origin. Typically such cells, when isolated, retain two or more mesodermal or mesenchymal developmental phenotypes (i.e., they are pluripotent). In particular, such cells generally have the capacity to develop into mesodermal tissues, such as mature adipose tissue, bone, various tissues of the heart (e.g., pericardium, epicardium, epimyocardium, myocardium, pericardium, valve tissue, etc.), dermal connective tissue, hemangial tissues (e.g., corpuscles, endocardium, vascular epithelium, etc.), muscle tissues (including skeletal muscles, cardiac muscles, smooth muscles, etc.), urogenital tissues (e.g., kidney, pronephros, meta- and meso-nephric ducts, metanephric diverticulum, ureters, renal pelvis, collecting tubules, epithelium of the female reproductive structures (particularly the oviducts, uterus, and vagina)), pleural and peritoneal tissues, viscera, mesodermal glandular tissues (e.g., adrenal cortex tissues), and stromal tissues (e.g., bone marrow). Of course, inasmuch as the cell can retain potential to develop into mature cells, it also can realize its developmental phenotypic potential by differentiating into an appropriate precursor cell (e.g., a preadipocyte, a premyocyte, a preosteocyte, etc.). Also, depending on the culture conditions, the cells can exhibit developmental phenotypes such as embryonic, fetal, hematopoetic, neurogenic, or neuralgiagenic developmental phenotypes. In this sense, stem cells can have two or more developmental phenotypes such as adipogenic, chondrogenic, cardiogenic, dermatogenic, hematopoetic, hemangiogenic, myogenic, nephrogenic, neurogenic, neuralgiagenic, urogenitogenic, osteogenic, pericardiogenic, peritoneogenic, pleurogenic, splanchogenic, and stromal developmental phenotypes. While such cells can retain two or more of these developmental phenotypes, preferably, such cells have three or more such developmental phenotypes (e.g, four or more mesodermal or mesenchymal developmental phenotypes), and some types of inventive stem cells have a potential to acquire any mesodermal phenotppe through the process of differentiation.

The cells have been characterized as to several of their cellular, genetic, immunological, and biochemical properties. For example, the cells have been characterized by their growth, by their cell surface markers, by their gene expression, by their ability to produce certain biochemical trophic factors, and by their immunological properties.

### Derivation and Expansion of Stem Cells

In one embodiment, SCPs are derived from pluripotent human cells (hEG) exhibiting an embryonic cell phenotype. The starting material for isolating the cells may be primordial germ cells isolated over a period of about 3 to about 13 weeks post-fertilization, or more preferably from about 5 to about 9 weeks post-fertilization, from embryonic yolk sac, mesenteries, or gonadal ridges, successively from human embryos/fetus. In another embodiment, gonocytes of later testicular stages are isolated. The primordial germ cells (PGCs) are cultured on mitotically inactivated fibroblast cells (e.g., STO cells) under conditions in long term cell culture (more than 30 days) to allow the production of hEGs. The resulting cells resemble human ES cells in morphology and in biochemical histotype. The cells can be passaged, maintained for several months in culture and survive cryopreservation.

The hEG stains positively for the presence of alkaline phosphatase (AP), therefore, AP is one measurement that can be used to identify hEG cells. The hEG cells also expresses cell surface antigens SSEA-1 and SSEA-4 and express cell surface antigens that bind to antibodies having the binding specificity of monoclonal antibodies TRA-1-60 and TRA-1-81. hEGs of the invention can also express the cell surface antigen SSEA-3. Depending upon the culture conditions, the hEG can differentiate into a variety of mature adult cell phenotypes that stain positively for particular biochemical markers and do not stain for other biochemical markers. Differentiated hEGs also exhibit, in still another embodiment, mature morphological features that enable one skilled in the art to distinguish them from non-differentiated hEGs.

The term “culture medium” means a suitable medium capable of supporting growth of cells. Examples of suitable culture media useful in practicing the present invention are a variety of hEG growth media prepared with a base of Dulbecco's minimal essential media (DMEM) supplemented with 15% fetal calf serum, 2 mM glutamine, 1 mM sodium pyruvate, or glucose and phosphate free modified human tubal fluid media (HTF) supplemented with 15% fetal calf serum, 0.2 mM glutamine, 0.5 mM taurine, and 0.01 mM each of the following amino acids; asparagine, glycine, glutamic acid, cysteine, lysine, proline, serine, histidine, and aspartic acid (McKiernan, S. M. Clayton, and B. Bavister, Molecular Reproduction and Development 42: 188-199, 1995). An effective amount of factors are then added daily to either of these base solutions to prepare hEG growth media.

One class of factors are ligands for receptors that activate the signal transduction gp130, either by binding to a receptor that heterodimerizes with gp130 or by binding directly to and activating gp130. For example, human recombinant leukemia inhibitory factor (LIF) at about 1000 U/ml to 2000 U/ml or oncostatin-M at 10 U/ml, can be used (Koshimizu, U., et al., Development 122: 1235-1242, 1996).

A second class of factors are those which elevate intracellular cAMP levels. For example, one or more of the following factors can be used at the stated final concentration: forskolin at 10 micromolar, cholera toxin at 10 micromolar, isobutylmethylxanthine (IBMX) at 0.1 mM, dibutyrladenosine cyclic monophosphate (dbcAMP) at 1 mM (Dolci, S., M. Pesce, and M. De Felici, Molecular Reproduction and Development 35: 134-139, 1993; De Felici, M., S. Dolci, and M. Pesce, Developmental Biology 157: 277-280, 1993; Halaban, R., et al., 1993).

A third class of factors are growth factors. In one particular embodiment the growth factor is basic fibroblast growth factor (bFGF), and more specifically, human recombinant basic fibroblast growth factor (bFGF) in the range of about 1 to about 10 ng/ml.

A fourth factor is growth media harvested from the culture of human embryonal carcinoma (EC) cells. In one embodiment, for example, human NTERA-2 EC cells (ATCC accession number CRL 1973) are grown to confluence in DMEM supplemented with 10% fetal calf serum or mouse ES cells are grown to confluence in DMEM supplemented with 15% fetal calf serum, 2 mM glutamine, 1000 U/ml LIF. Growth media is harvested daily over several days, passed through a 0.22 micron filter and frozen at −80° C. This EC or ES “conditioned” media is added to the hEG growth media in empirically determined amounts, as judged by the effect on hEG growth and viability.

The term “STO cell” refers to embryonic fibroblast mouse cells such as are commercially available and include those deposited as ATCC CRL 1503, and ATCC 56-X. After the hEG cells are isolated, they can be maintained by methods of growth and maintenance of cells known in the art. It is understood that other fibroblast cells can be used as long as they can function as feeder cells for the production of hEG cells of the invention.

In another embodiment, SCPs are derived from human mesenchymal stem cells obtained from the bone marrow or other mesenchymal stem cell source. Bone marrow cells may be obtained from iliac crest, femora, tibiae, spine, rib or other medullary spaces. Other sources of human mesenchymal stem cells include embryonic yolk sac, fetal and adolescent skin, and blood.

These cells can be culturally expanded, for example, in BGJb medium containing 10% fetal serum or in a chemically defined medium that does not require serum. Suitable media for culture expansion of these cells are described in U.S. Pat. No. 5,486,359, issued Jan. 23, 1996, and suitable chemically defined media which do not require the presence of serum are described in U.S. application Ser. No. 08/464,599, filed Jun. 5, 1995.

SCPs can be derived from stem cells obtained from adipose tissue by any suitable method. A first step in any such method requires the isolation of adipose tissue from the source donor. The donor can be alive or cadaveric. Typically, human adipose stromal cells are obtained from living donors, using well-recognized protocols such as surgical or suction lipectomy. Indeed, as liposuction procedures are so common, liposuction effluent is a preferred source from which the adipose-derived stem cells for use in SCPs are derived.

However derived, the adipose tissue is processed to separate stem cells from the remainder of the material. In one protocol, the adipose tissue is washed with physiologically-compatible saline solution (e.g., phosphate buffered saline (PBS)) and then vigorously agitated and left to settle, a step that removes loose mater (e.g., damaged tissue, blood, erythrocytes, etc.) from the adipose tissue. Thus, the washing and settling steps generally are repeated until the supernatant is relatively clear of debris.

Following the final isolation and resuspension, the adipose-derived stem cells can be cultured and, if desired, assayed for number and viability to assess the yield. Desirably, the cells can be cultured without differentiation using standard cell culture media (e.g, DMEM, typically supplemented with 5-15% serum (e.g., fetal bovine serum, horse serum, etc.). Preferably, the cells can be passaged at least five times in such medium without differentiating, while still retaining their developmental phenotype, and more preferably, the cells can be passaged at least 10 times (e.g., at least 15 times or even at least 20 times) without losing developmental phenotype. Thus, culturing the cells of the present invention without inducing differentiation can be accomplished without specially screened lots of serum, as is generally the case for mesenchymal stem cells (e.g., derived from marrow). Methods for measuring viability and yield are known in the art (e.g., trypan blue exclusion).

In one embodiment, SCPs are derived from hematopoietic progenitors expressing high levels of the cell surface glycoprotein CD34. CD34+ cells are capable of initiating long-term hematopoiesis both in vitro and in vivo. CD34+ progenitors can be derived from Bone Marrow (in a non-limiting example, obtained from normal donors by bilateral aspirates of the posterior iliac crest), Mobilized Peripheral Blood (in a non-limiting example, progenitors are mobilized into the bloodstream of the donor by daily injections of G-CSF (7.5 microgram/kg) for 4 days followed by apheresis on day 5 to harvest mononuclear cells enriched with progenitors), Umbilical Cord Blood, and Fetal Liver. CD34+ progenitors are isolated from mononuclear cells using positive immunomagnetic selection. Culture media used to expand hematopoietic CD34+ progenitors include, but are not limited to DPBM, and IMDM+15% FBS. There are a variety of growth factors that may be used including G-CSF, GM-SCF and SCF. Multiple growth factors may be required for optimum growth.

In another embodiment, SCPs are derived from CD133+ progenitor cells isolated from human bone marrow, G-CSF mobilized peripheral blood, umbilical cord blood, and fetal liver by positive immunoselection. Culture media used to expand hematopoietic CD133+ progenitors include, but are not limited to DPBM, and IMDM+15% FBS. There are a variety of growth factors that may be used including G-CSF, GM-SCF and SCF. Multiple growth factors may be required for optimum growth.

### Production of Stem Cell Products (SCPs)

In one embodiment, whole cell lysates are prepared, e.g., by disrupting cells without subsequent separation of cell fractions. In another embodiment, a cell membrane fraction is separated from a soluble fraction of the cells by routine methods known in the art, e.g., centrifugation, filtration, or similar methods. Methods of lysing cells are well-known in the art and include various means of freeze-thaw disruption, osmotic disruption, mechanical disruption, ultrasonic disruption, enzymatic disruption (e.g., hyaluronidase, dispase, proteases, and nucleases (for example, deoxyribonuclease and ribonuclease)), or chemical disruption (non-ionic detergents such as, alkylaryl polyether alcohol (TRITON® X-100), octylphenoxy polyethoxy-ethanol (Rohm and Haas, Philadelphia, Pa.), BRIJ-35, a polyethoxyethanol lauryl ether (Atlas Chemical Co., San Diego, Calif.), polysorbate 20 (TWEEN 20®), a polyethoxyethanol sorbitan monolaureate (Rohm and Haas), polyethylene lauryl ether (Rohm and Haas); and ionic detergents such as, for example, sodium dodecyl sulphate, sulfated higher aliphatic alcohols, sulfonated alkanes and sulfonated alkylarenes containing 7 to 22 carbon atoms in a branched or unbranched chain), or combinations thereof. Such cell lysates may be prepared from cells directly in their growth medium and thus containing secreted growth factors and the like, or may be prepared from cells washed free of medium in, for example, PBS or other solution. Cells may also be lysed on their growth substrate. Washed cells may be resuspended at concentrations greater than the original population density if preferred. Cell lysates prepared from populations of stem cells may be used as is, further concentrated, by for example, ultrafiltration or lyophilization, or even dried, partially purified, combined with pharmaceutically acceptable carriers or diluents as are known in the art, or combined with other compounds such as biologicals, for example pharmaceutically useful protein compositions. In some embodiments, cellular membranes are removed from the lysate, for example by centrifugation, or ultracentrifugation, filtration, chromatograph, or sedimentation, to yield a membrane fraction and supernate fraction. The membrane fraction or the supernate may be used according to the methods of the invention. In some embodiments, the whole cell lysate or a cell fraction can be processed with molecular weight cut off filters to obtain a lysate of a particular molecular weight including, but not limited to 5,000 to 100,000 kDa. SCP filtered to obtained fractions of a defined molecular weight range can be combined with other SCP filtered fractions to obtain other specific defined molecular weight ranges. In some embodiments, cellular debris is removed by treatment with a mild detergent rinse, such as EDTA, CHAPS or a zwitterionic detergent. Cell lysates may be used in vitro or in vivo, alone or, for example, with cells or on a substrate. The cell lysates, if introduced in vivo, may be introduced locally at a site of treatment, or remotely to provide, for example needed cellular growth factors to a patient.

### Characterization of SCPs

SCPs may be characterized, for example by protein quantification (e.g. colormetric assays including, but not limited to BCA (bicinchonic acid) assay, Braford assay, Lowry assay, Modified Lowry assay, Biuret, Amido black method, and colloidal gold), protein electrophoresis (including, but not limited to Polyacrylamide gel elctrophoresis (PAGE), first dimensional (isoelectric focusing) protein separation, second dimensional (2D) protein speration by vertical gel elctrophoresis systems of minigel systems) used in combination with stains (including, but not limited to silver stain markers, fluorescent markers, biotinulatred markers, and recombinate markers), mass spectroscopy (including, but not limited to LC-MS, MALDI-TOF, Q-TOF, Ion trap-MS) chromatography (including, but not limited to HPLC, affinity chromatography, gel filtration chromotogaphy, and ion exchange chromatography) X-ray crystallography, and antibody based assay systems (including, but not limited to Western blot analysis, ELISA, Multiplex ELISA, and Proteomics) and in vitro biological potency and release assays (including, but not limited to cell proliferation asssays and transwell assays).

In some embodiments, SPCs can be characterized by total protein content. As a non-limiting example SCP derived from the lysis and centrifugational separation of hUTC yield a total protein per cell ranging from 12 to 130 pg with an average of about 57.5 pg which correlates to the cell density at harvest. As an additional non-limiting example SCP derived from the lysis and centrifugational separation of hMSC yield a total protein per cell range of 10 to 200 pg with an average of about 107 pg.

In one embodiment, SPC can be assayed by ELISA for the presence of growth factors including, but not limited to bFGF. As a non-limiting example SCP derived from the lysis and centrifugational separation of hUTC yield a bFGF per cell ranging from 1.49 to 4.37 pg with an average of 3.09. In a further embodiment bFGF(pg/ml)=(28.745)total cell protein(micrograms/ml)−25.656.

In additional embodiments, SPC can by assayed by Multi-Plex ELISA for growth factors, cytokines, and other therapeutic factors including, but not limited to KGF, PDGF-BB, HGF, TGF-alpha, BDNF, and IL-6. As a non-limiting example SCP derived from the lysis and centrifugational separation of hUTC assayed by Multi-Plex ELISA showed significant levels of KGF, PDGF-BB, HGF, TGF-alpha, BDNF, and IL-6 present in the SCP.

In preferred embodiments, SCP is assayed in vitro for its biological efficacy using cell proliferation asssays. SCP supplemented into cell culture media will increase proliferation in the target cell relative to the vehicle control over time. Target cell types include, but are not limited to NIH/3T3 fibroblasts, epithelial cells (e.g., cells of oral mucosa, gastrointestinal tract, nasal epithelium, respiratory tract epithelium, vaginal epithelium, corneal epithelium), bone marrow cells, adipocytes, stem cells, keratinocytes, melanocytes, dermal fibroblasts, vascular endothelial cells (e.g., aortic endothelial cells, coronary artery endothelial cells, pulmonary artery endothelial cells, iliac artery endothelial cells, microvascular endothelial cells, umbilical artery endothelial cells, umbilical vein endothelial cells, and endothelial progenitors (e.g., CD34+, CD34+/CD 117+ cells)), myoblasts, myocytes, stromal cells, and other soft tissue cells or progenitor cells.

In additional embodiments, SCP combined with a matrix is assayed in vitro for its biological efficacy and release kintics using a transwell cell proliferation asssays. SCP combined with a matrix and incubated in the top portion of a transwell system will increase proliferation in the target cell relative to the vehicle control over time. Target cells type include but are not limited to NIH/3T3 fibroblasts, epithelial cells (e.g., cells of oral mucosa, gastrointestinal tract, nasal epithelium, respiratory tract epithelium, vaginal epithelium, corneal epithelium), bone marrow cells, adipocytes, stem cells, keratinocytes, melanocytes, dermal fibroblasts, vascular endothelial cells (e.g., aortic endothelial cells, coronary artery endothelial cells, pulmonary artery endothelial cells, iliac artery endothelial cells, microvascular endothelial cells, umbilical artery endothelial cells, umbilical vein endothelial cells, and endothelial progenitors (e.g., CD34+, CD34+/CD117+ cells)), myoblasts, myocytes, stromal cells, and other soft tissue cells or progenitor cells.

### Methods of Using SCPs

### Therapeutic Applications of SCP

SCPs may be used to treat patients having a soft tissue condition, including, but not limited to patients requiring the repair or replacement of soft tissue resulting from disease or trauma or failure of the tissue to develop normally, or to provide a cosmetic function, such as to augment features of the body. The treatment may comprise at least one of soft tissue repair, reconstruction, bulking, cosmetic treatment, therapeutic treatment, tissue augmentation, and tissue sealing. Provided herein are methods of treating soft tissue conditions in a patient by administering to the patient SCP. Therapeutic applications of the SCP include, but are not limited to treatment of hernias, congenital defects, damage to the pelvic floor, tear or rupture of a tendon or ligament, a traumatic wound, skin repair and regeneration (e.g., scar revision or the treatment of traumatic wounds, burns, skin ulcers (e.g., decubitus (pressure) ulcers, venous ulcers, and diabetic ulcers), surgical wounds such as those associated with the excision of skin cancers; treatment of vascular conditions (e.g., vascular disease such as peripheral arterial disease, abdominal aortic aneurysm, carotid disease, and venous disease; vascular injury; improper vascular development); muscle diseases (e.g., congenital myopathies; myasthenia gravis; inflammatory, neurogenic, myogenic muscle diseases; and muscular dystrophies such as Duchenne muscular dystrophy, Becker muscular dystrophy, myotonic dystrophy, limb-girdle-muscular dystrophy, facioscapulohumeral muscular dystrophy, congenital muscular dystrophies, oculopharyngeal muscular dystrophy, distal muscular dystrophy, and Emery-Dreifuss muscular dystrophy), breast reconstruction, and bladder augmentation.

Also provided by the invention are methods of treating a patient in need of angiogenic factors comprising administering to the patient the SCP of the invention.

SCP of the invention may be administered alone or as admixtures with other cells. For example, SCP may be administered by way of a matrix. A matrix may comprise a three-dimensional scaffold. Scaffolds may be particulate, flat, tubular, single-layered, or multilayered. The SCP may be administered with conventional pharmaceutically acceptable carriers. Where SCPs are to be administered with other cells, the SCPs may be administered simultaneously or sequentially with the other cells. Where cells are to be administered sequentially with other cell types, the SCPs may be administered before or after the cells. Cells which may be administered in conjunction with SCP include epithelial cells (e.g., cells of oral mucosa, gastrointestinal tract, nasal epithelium, respiratory tract epithelium, vaginal epithelium, corneal epithelium), bone marrow cells, adipocytes, stem cells, keratinocytes, vascular endothelial cells (e.g., aortic endothelial cells, coronary artery endothelial cells, pulmonary artery endothelial cells, iliac artery endothelial cells, microvascular endothelial cells, umbilical artery endothelial cells, umbilical vein endothelial cells, and endothelial progenitors (e.g., CD34+, CD34+/CD117+ cells)), myoblasts, myocytes, stromal cells, bladder urothelial cells, smooth muscle cells, gastrointestinal cells, esophageal cells, larynx cells, mucosal cells, and other soft tissue cells or progenitor cells.

The SCP may be administered with other beneficial drugs or biological molecules (e.g., growth factors, trophic factors). The pharmaceutical compositions of the invention comprise SCP and a pharmaceutically acceptable carrier. In preferred embodiments, the pharmaceutical compositions comprise SCP in a therapeutically effective amount sufficient to treat a soft tissue condition. When administered with other agents, the SCP may be administered together in a single pharmaceutical composition, or in separate pharmaceutical compositions, simultaneously or sequentially with the other bioactive factors (either before or after administration of the other agents). Bioactive factors which may be co-administered include anti-apoptotic agents (e.g., EPO, EPO mimetibody, TPO, IGF-I and IGF-II, HGF, caspase inhibitors); anti-inflammatory agents (e.g., p38 MAPK inhibitors, TGF-beta inhibitors, statins, IL-6 and IL-1 inhibitors, pemirolast, tranilast, REMICADE, and NSAIDs (non-steroidal anti-inflammatory drugs; e.g., tepoxalin, tolmetin, suprofen); immunosupressive/immunomodulatory agents (e.g., calcineurin inhibitors, such as cyclosporine, tacrolimus; mTOR inhibitors (e.g., sirolimus, everolimus); anti-proliferatives (e.g., azathioprine, mycophenolate mofetil); corticosteroids (e.g., prednisolone, hydrocortisone); antibodies such as monoclonal anti-IL-2Ralpha receptor antibodies (e.g., basiliximab, daclizumab), polyclonal anti-T-cell antibodies (e.g., anti-thymocyte globulin (ATG); anti-lymphocyte globulin (ALG); monoclonal anti-T cell antibody OKT3)); anti-thrombogenic agents (e.g., heparin, heparin derivatives, urokinase, PPack (dextrophenylalanine proline arginine chloromethylketone), antithrombin compounds, platelet receptor antagonists, anti-thrombin antibodies, anti-platelet receptor antibodies, aspirin, dipyridamole, protamine, hirudin, prostaglandin inhibitors, and platelet inhibitors); and anti-oxidants (e.g., probucol, vitamin A, ascorbic acid, tocopherol, coenzyme Q-10, glutathione, L-cysteine, N-acetylcysteine) as well as local anesthetics. As another example, the cells may be co-administered with scar inhibitory factor as described in U.S. Pat. No. 5,827,735, incorporated herein by reference.

Pharmaceutical compositions of the invention may comprise, in addition to the SCP, at least one cell type. For example, pharmaceutical compositions of the invention may comprise a soft tissue cell. Examples of the at least one other cell type to be included in the pharmaceutical compositions of SCP of the invention include stem cells, epithelial cells, dermal fibroblasts, melanocytes, keratinocytes, and other epithelial progenitor cells, myocytes, myoblasts, and muscle cells (e.g., smooth muscle cells), endothelial cells, and stromal cells.

The SCP and related products of the invention may be surgically implanted, injected, engrafted, delivered (e.g., by way of a catheter or syringe), or otherwise administered directly or indirectly to the site of soft tissue condition. SCP may be administered by way of a matrix (e.g., a three-dimensional scaffold), or via injectable viscoelastic supplements such as hyaluronic acid, alginates, self-assembling peptides, hydrogels and collagen. SCP may be administered with conventional pharmaceutically acceptable carriers. Routes of administration of SCP include intramuscular, intravenous, intraarterial, intraperitoneal, subcutaneous, oral, and nasal administration. Preferable routes of in vivo administration include transplantation, implantation, injection, delivery via a catheter, microcatheter, suture, stent, microparticle, pump, or any other means known in the art.

When SCPs are administered in semi-solid or solid devices, surgical implantation into a precise location in the body is typically a suitable means of administration.

Dosage forms and regimes for administering SCP described herein are developed in accordance with good medical practice, taking into account the condition of the individual patient, e.g., nature and extent of the condition being treated, age, sex, body weight and general medical condition, and other factors known to medical practitioners. Thus, the effective amount of a pharmaceutical composition to be administered to a patient is determined by these considerations as known in the art.

### Compositions and Pharmaceutical Compositions

Compositions of SCP (e.g., cell fraction, secreted factors), including for example pharmaceutical compositions, are included within the scope of the invention. Compositions of the invention may include one or more bioactive factors, including, but not limited to a growth factor, a differentiation-inducing factor, a cell survival factor such as caspase inhibitor, an anti-inflammatory agent such as p38 kinase inhibitor, or an angiogenic factor such as VEGF or bFGF. More examples of bioactive factors include PDGF-bb, EGF, bFGF, IGF-1, and LIF.

Pharmaceutical compositions of the invention may also comprise homogeneous or heterogeneous populations of differentiated and/or undifferentiated stem cells in a pharmaceutically acceptable carrier.

Pharmaceutically acceptable carriers include organic or inorganic carrier substances suitable that do not deleteriously react with the SCP of the invention or related products. To the extent they are biocompatible, suitable pharmaceutically acceptable carriers include water, salt solution (such as Ringer's solution), alcohols, oils, gelatins, and carbohydrates, such as lactose, amylose, or starch; fatty acid esters, hydroxymethylcellulose, hyaluronic acid, and polyvinyl pyrolidine. Such preparations can be sterilized, and if desired, mixed with auxiliary agents such as lubricants, preservatives, stabilizers, wetting agents, emulsifiers, salts for influencing osmotic pressure, buffers, and coloring. Pharmaceutical carriers suitable for use in the present invention are known in the art and are described, for example, in Pharmaceutical Sciences (17^{th} Ed., Mack Pub. Co., Easton, Pa.) and WO 96/05309, each of which are incorporated by reference herein.

The compositions may be delivered in the form of a spray, suspension, solution, dry powder, cream, ointment, or gel.

The dosage (e.g., the mass of SCP to be administered) and frequency of administration of the pharmaceutical compositions will depend upon a number of factors including, but not limited to the nature of the condition to be treated, the extent of the symptoms of the condition, and the characteristics of the patient (e.g., age, size, gender, health).

For example, but not by way of limitation, SCPs, matrices, vascular networks, and compositions produced according to the invention may be used to repair or replace underdeveloped, damaged, or destroyed soft tissue, to augment existing soft tissue, to introduce new or altered tissue, to modify artificial prostheses, or to join biological tissues or structures. Some embodiments soft tissue conditions include (i) hernia closures with replacement soft tissue constructs grown in three-dimensional cultures; (ii) skin grafts with soft tissue constructs; (iii) prostheses; (iv) blood vessel grafts; and (v) tendon or ligament reconstruction. Examples of such conditions that can be treated according to the methods of the invention include congenital anomalies such as hemifacial microsomia, malar and zygomatic hypoplasia, unilateral mammary hypoplasia, pectus excavatum, pectoralis agenesis (Poland's anomaly) and velopharyngeal incompetence secondary to cleft palate repair or submucous cleft palate (as a retropharyngeal implant); acquired defects (post-traumatic, post-surgical, post-infectious) such as scars, subcutaneous atrophy (e.g., secondary to discoid lupus erythematosus), keratotic lesions, acne pitting of the face, linear scleroderma with subcutaneous atrophy, saddle-nose deformity, Romberg's disease, and unilateral vocal cord paralysis; cosmetic defects such as glabellar frown lines, deep nasolabial creases, circum-oral geographical wrinkles, sunken cheeks and mammary hypoplasia; hernias; tears or ruptures of a tendon or ligament; severe burns, skin ulcers (e.g., decubitus (pressure) ulcers, venous ulcers, and diabetic ulcers), and surgical wounds such as those associated with the excision of skin cancers; vascular diseases such as peripheral arterial disease, abdominal aortic aneurysm, carotid disease, and venous disease; muscle diseases (e.g., congenital myopathies; myasthenia gravis; inflammatory, neurogenic, and myogenic muscle diseases; and muscular dystrophies such as Duchenne muscular dystrophy, Becker muscular dystrophy, myotonic dystrophy, limb-girdle-muscular dystrophy, facioscapulohumeral muscular dystrophy, congenital muscular dystrophies, oculopharyngeal muscular dystrophy, distal muscular dystrophy, and Emery-Dreifuss muscular dystrophy); and replacement and repair of connective tissues such as tendons and ligaments.

The successful repair or replacement of damaged tissue can be enhanced if the implanted cells and/or tissue can be fixed in place at the site of repair. Post-implantation movement may cause the new cells or tissue to become dislodged from the site if a pro-active fixation technique is not employed. Various methods can be used to fix the new cells and/or tissue in place, including: patches derived from biocompatible tissues, which can be placed over the site; biodegradable sutures, hollow sutures, porous sutures, or other fasteners, e.g., pins, staples, tacks, screws and anchors; non-absorbable fixation devices, e.g., sutures, pins, screws and anchors; adhesives; and the use of interference fit geometries.

The SCP of the invention may be administered alone, in a pharmaceutically acceptable carrier, through a catheter or microcatheter, via a pump or spray, or on or in a matrix as described herein.

### Use of SCP for Transplantation

In an embodiment, a formulation comprising SCP is prepared for administration directly to the site where the new soft tissue is desired. In some embodiments, the support for the SCP of the invention is biodegradable. As an example of a formulation of the invention, and not by way of limitation, SCP of the invention may be suspended in a hydrogel solution for injection. Examples of suitable hydrogels for use in the invention include self-assembling peptides, such as RAD16. Alternatively, the hydrogel solution may be allowed to set, for instance in a mold, to form a matrix having SCP dispersed therein prior to implantation. Or, once the matrix has set, the cell formulations may be cultured so that the cells are mitotically expanded prior to implantation. Hydrogels are organic polymers (natural or synthetic) that are cross-linked via covalent, ionic, or hydrogen bonds to create a three-dimensional open-lattice structure which entraps water molecules to form a gel. Examples of materials which can be used to form a hydrogel include polysaccharides such as alginate and salts thereof, peptides, polyphosphazines, and polyacrylates, which are crosslinked ionically, carboxymethyl cellulose (CMC), oxidized regenerated cellulose (ORC), or block polymers such as polyethylene oxide-polypropylene glycol block copolymers which are crosslinked by temperature or pH, respectively. In some embodiments of the invention, the formulation comprises an in situ polymerizable gel, as described, for example, in U.S. Patent Application Publication 2002/0022676; Anseth et al., J. Control Release, 78(1-3): 199-209 (2002); Wang et al., Biomaterials, 24(22):3969-80 (2003). Methods of synthesis of the hydrogel materials, as well as methods for preparing such hydrogels, are known in the art.

Other components may also be included in the formulation, including but not limited to any of the following: (1) buffers to provide appropriate pH and isotonicity; (2) lubricants; (3) viscous materials to retain the cells at or near the site of administration, including, for example, alginates, agars and plant gums; and (4) other cell types that may produce a desired effect at the site of administration, such as, for example, enhancement or modification of the formation of tissue or its physicochemical characteristics, or as support for the viability of the cells, or inhibition of inflammation or rejection. The cells may be covered by an appropriate wound covering to prevent cells from leaving the site. Such wound coverings are known to those of skill in the art.

Bioactive factors which may be usefully incorporated into the compositions of the invention include anti-apoptotic agents (e.g., EPO, EPO mimetibody, TPO, IGF-I and IGF-II, HGF, caspase inhibitors); anti-inflammatory agents (e.g., p38 MAPK inhibitors, TGF-beta inhibitors, statins, IL-6 and IL-1 inhibitors, pemirolast, tranilast, REMICADE, and NSAIDs (non-steroidal anti-inflammatory drugs; e.g., tepoxalin, tolmetin, suprofen); immunosupressive/immunomodulatory agents (e.g., calcineurin inhibitors, such as cyclosporine, tacrolimus; mTOR inhibitors (e.g., sirolimus, everolimus); anti-proliferatives (e.g., azathioprine, mycophenolate mofetil); corticosteroids (e.g., prednisolone, hydrocortisone); antibodies such as monoclonal anti-IL-2Ralpha receptor antibodies (e.g., basiliximab, daclizumab), polyclonal anti-T-cell antibodies (e.g., anti-thymocyte globulin (ATG); anti-lymphocyte globulin (ALG); monoclonal anti-T cell antibody OKT3)); anti-thrombogenic agents (e.g., heparin, heparin derivatives, urokinase, PPack (dextrophenylalanine proline arginine chloromethylketone), antithrombin compounds, platelet receptor antagonists, anti-thrombin antibodies, anti-platelet receptor antibodies, aspirin, dipyridamole, protamine, hirudin, prostaglandin inhibitors, and platelet inhibitors); and anti-oxidants (e.g., probucol, vitamin A, ascorbic acid, tocopherol, coenzyme Q-10, glutathione, L-cysteine, N-acetylcysteine) as well as local anesthetics. As another example, the cells may be co-administered with scar inhibitory factor as described in U.S. Pat. No. 5,827,735, incorporated herein by reference.

### Transplantation of SCP Using Scaffolds

The SCP may be combined/incorporated onto a scaffold, such as a three-dimensional scaffold, and implanted in vivo, where the SCP will elute over time or in a burst from the scaffold, induce tissue repair and regeneration within the scaffold and its periphery, and form a replacement tissue in vivo in cooperation with the cells of the patient.

For examples the scaffolds of the invention can be used to form tubular structures, like those of the gastrointestinal and genitourinary tracts, as well as blood vessels; tissues for hernia repair; ten dons and ligaments.

Some embodiments of the invention provide a matrix for implantation into a patient. The matrix may also be inoculated with cells of another desired cell type, for example but not by way of limitation, epithelial cells (e.g., cells of oral mucosa, gastrointestinal tract, nasal epithelium, respiratory tract epithelium, vaginal epithelium, corneal epithelium), bone marrow cells, adipocytes, stem cells, keratinocytes, melanocytes, dermal fibroblasts, vascular endothelial cells (e.g., aortic endothelial cells, coronary artery endothelial cells, pulmonary artery endothelial cells, iliac artery endothelial cells, microvascular endothelial cells, umbilical artery endothelial cells, umbilical vein endothelial cells, and endothelial progenitors (e.g., CD34+, CD34+/CD117+ cells)), myoblasts, myocytes, stromal cells, and other soft tissue cells or progenitor cells. The matrix may contain or be pre-treated with one or more bioactive factors including, for example, drugs, anti-inflammatory agents, antiapoptotic agents, and growth factors. In some embodimetns, the matrix is inoculated with SCP, including for example, secreted factors or cell fractions of the SCPs. In some embodiments, the matrix is biodegradable. In some embodiments, the matrix comprises extracellular membrane proteins, for example, MATRIGEL. In some aspects of the invention, the matrix comprises natural or synthetic polymers. Matrices of the invention include biocompatible scaffolds, lattices, self-assembling structures and the like, whether biodegradable or not, liquid or solid. Such matrices are known in the arts of cell-based therapy, surgical repair, tissue engineering, and wound healing. Preferably the matrices are pretreated (e.g., seeded, inoculated, contacted with) with SCP (e.g., secreted factors, cell fraction, or combination thereof) of the invention. More preferably, SCPs are in close association to the matrix or its spaces. In some aspects of the invention, SCPs adhere to the matrix. In some embodiments, SCPs are contained within or bridge interstitial spaces of the matrix. Most preferred are those matrices wherein SCPs are in close association with the matrix and which, when used therapeutically, induce or support ingrowth of the patient's cells and/or proper angiogenesis. The SCP pre-treated matrices can be introduced into a patient's body in any way known in the art, including but not limited to implantation, injection, surgical attachment, transplantation with other tissue, and the like. The matrices of the invention may be configured for use in vivo, for example, to the shape and/or size of a tissue or organ in vivo. The scaffolds of the invention may be flat or tubular or may comprise sections thereof, as described herein. The scaffolds of the invention may be multilayered.

For example, but not by way of limitation, the scaffold may be designed such that the scaffold structure: (1) supports the SCP without subsequent degradation; or (2) supports the SCP from the time of combination to the scaffold until the scaffold is remodeled by the host tissue. A review of scaffold design is provided by Hutmacher, J. Biomat. Sci. Polymer Edn., 12(1):107-124 (2001).

Scaffolds of the invention can be administered in combination with any one or more growth factors, cells, drugs, or other components described above that stimulate soft tissue formation or stimulate vascularization or innervation thereof or otherwise enhance or improve the practice of the invention.

In some embodiments, it is important to re-create in culture the cellular microenvironment found in vivo, such that the extent to which the SCP of the invention are combined prior to in vivo administration or use in vitro may vary. SCPs may be combined onto the scaffold before or after forming the desired shape, e.g., ropes, tubes, filaments.

Examples of scaffolds which may be used in the present invention include nonwoven or woven mats, porous foams, sutures, beads, microparticles, or hydrogels. Nonwoven mats may, for example, be formed using fibers comprised of poly(lactic acid-co-glycolic acid) polymer (10/90 PLGA), referred to herein as VNW, available for purchase through Biomedical Structures (Slatersville, R.I.). Foams, composed of, for example, poly(epsilon-caprolactone)/poly(glycolic acid) (PCL/PGA) copolymer, formed by processes such as freeze-drying, or lyophilization, as discussed in U.S. Pat. No. 6,355,699, are also possible scaffolds. Hydrogels such as self-assembling peptides (e.g., RAD16) may also be used. Another embodiment of a scaffold or matrix of the invention comprises collagen/ORC, CMC, or ORC. These materials are frequently used as supports for growth of tissue. In some embodiments, the scaffold is lyophilized prior to use. In some embodiments, lyophilized scaffolds are rehydrated, with saline for example, prior to use. According to a preferred embodiment, the scaffold is a felt, which can be composed of a multifilament yarn made from a bioabsorbable material, e.g., PGA, PLA, PCL copolymers or blends, or hyaluronic acid. The yarn is made into a felt using standard textile processing techniques consisting of crimping, cutting, carding and needling.

In another embodiment, SCPs are combined with foam scaffolds that may be composite structures. In addition, the three-dimensional scaffold may be molded into a useful shape, such as a specific structure in the body to be repaired, replaced, or augmented.

The scaffold may be treated prior to combination to enhance attachment of the SCP. For example, prior to combination, nylon matrices could be treated with 0.1 molar acetic acid and incubated in polylysine, PBS, and/or collagen to coat the nylon. Polystyrene could be similarly treated using sulfuric acid.

In addition, the external surfaces of the three-dimensional scaffold may be modified to improve the attachment or growth of cells and differentiation of tissue, such as by plasma coating the scaffold or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate), a cellular matrix, and/or other materials including, but not limited to, gelatin, alginates, agar, agarose, and plant gums, among others.

In some embodiments, the scaffold is comprised of or is treated with materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as ePTFE, and segmented polyurethaneurea silicones, such as PURSPAN (The Polymer Technology Group, Inc., Berkeley, Calif.). These materials can be further treated to render the scaffold non-thrombogenic. Such treatments include anti-thrombotic agents such as heparin, and treatments which alter the surface charge of the material such as plasma coating.

Different proportions of the various types of collagen, for example, deposited on the scaffold can affect the growth of tissue-specific or other cells which may be later inoculated onto the scaffold or which may grow onto the structure in vivo. For example, for three-dimensional skin culture systems, collagen types I and III are preferably deposited in the initial matrix. Alternatively, the scaffold can be inoculated with a mixture of cells which synthesize the appropriate collagen types desired. Thus, depending upon the tissue to be cultured, the appropriate collagen type to be inoculated on the scaffold or produced by the cells seeded thereon may be selected. For example, the relative amounts of collagenic and elastic fibers present in the scaffold can be modulated by controlling the ratio of collagen-producing cells to elastin-producing cells in the initial inoculum. For example, since the inner walls of arteries are rich in elastin, an arterial scaffold should contain a co-culture of smooth muscle cells which secrete elastin.

The SPC combined three-dimensional scaffold of the invention can be used in a variety of applications. These applications include but are not limited to transplantation or implantation of either cultured cells obtained from the matrix or the cultured matrix itself in vivo. The three-dimensional scaffolds may, according to the invention, be used to replace or augment existing tissue, to introduce new or altered tissue, to modify artificial prostheses, or to join together biological tissues or structures. For example, specific embodiments of the invention include but are not limited to, flat structures and tubular three-dimensional tissue implants for repair or regeneration, for example, of the gastrointestinal tract, genitourinary tract, blood vessels, muscles, ligaments, tendons, skin, pelvic floor, fascia, and hernias.

SCP can be combined onto a flat scaffold. Two or more flat scaffolds can be laid atop another and sutured together to generate a multilayer scaffold.

For example and not by way of limitation, the three-dimensional scaffold can be used to construct single and multi-layer tubular tissues in vitro that can serve as a replacement for damaged or diseased tubular tissue in vivo.

The following subsections describe the use of a seeded scaffold to prepare tubes comprising SCP and/or SCP products that can be implanted into the body.

A scaffold can be cut into a strip (e.g., rectangular in shape) of which the width is approximately equal to the inner circumference of the tubular organ into which it will ultimately be inserted. The cells can be inoculated onto the scaffold and incubated by floating or suspending in liquid media. At the appropriate stage of confluence, the scaffold can be rolled up into a tube by joining the long edges together. The seam can be closed by suturing the two edges together using fibers of a suitable material of an appropriate diameter.

According to the invention, a scaffold can be formed as a tube, and combined with SCP.

In general, two three-dimensional scaffolds can be combined into a tube in accordance with the invention using any of the following methods.

Two or more flat scaffolds can be laid atop another and sutured together. This two-layer sheet can then be rolled up, and, as described above, joined together and secured.

One tubular scaffold that is to serve as the inner layer can be combined with SCP. A second scaffold can be created as a flat strip with width slightly larger than the outer circumference of the tubular scaffold. The flat scaffold can be wrapped around the outside of the tubular scaffold followed by closure of the seam of the two edges of the flat scaffold and, preferably, securing the flat scaffold to the inner tube.

Two or more tubular meshes of slightly differing diameters can be created separately. The scaffold with the smaller diameter can be inserted inside the larger one and secured.

For each of these methods, more layers can be added by reapplying the method to the double-layered tube. Scaffolds comprising SCP may be layered with scaffolds comprising additional SCPs.

The lumenal aspect of the tubular construct can be comprised of or treated with materials that render the lumenal surface of the tubular scaffold non-thrombogenic. These treatments and materials may also promote and sustain endothelial growth, migration, and extracellular matrix deposition. Examples of these materials and treatments include, but are not limited to natural materials such as basement membrane proteins such as laminin and Type IV collagen, synthetic materials such as ePTFE, and segmented polyurethaneurea silicones, such as PURSPAN (The Polymer Technology Group, Inc., Berkeley, Calif.). These materials can be further treated to render the lumenal surface of the tubular scaffold non-thrombogenic. Such treatments include anti-thrombotic agents such as heparin, and treatments which alter the surface charge of the material such as plasma coating.

Advanced bioreactors may be necessary to meet the complex requirements of in vitro engineering of functional skeletal tissues. Bioreactor systems with the ability to apply complex concurrent mechanical strains to three-dimensional matrices, for example, in conjunction with enhanced environmental and fluidic control are provided by Altman et al., J. Biomech. Eng., 124(6):742-749 (2002); U.S. Patent Application Publication No. 2002/0062151. For example but not by way of limitation, such a bioreactor system may be used in the development of a tissue-engineered tendon or ligament, e.g., anterior cruciate ligament.

According to the present invention, any suitable method can be employed to shape the three-dimensional culture to assume the conformation of the natural organ or tissue to be simulated. For example, a scaffold prepared in accordance with the invention may be “trimmed” to a pre-selected size for surgical repair of the damaged tissue. Trimming may be performed with the use of a sharp cutting implement, i.e., a scalpel, a pair of scissors or an arthroscopic device fitted with a cutting edge, using procedures well known in the art.

The three-dimensional scaffolds can be shaped to assume a conformation which simulates the shape of a natural organ or tissue, such as soft tissue including but not limited to pelvic floor, bladder, fascia, skin, muscle, tendon, ligament, or vasculature (e.g., arteries, veins). These constructions simulate biological structures in vivo and may be readily implanted to repair hernias or to replace damaged or diseased tissues, including hernias, tendons, ligaments, skin, muscle, blood vessels, and components of the gastrointestinal tract, genitourinary tract (e.g., urethra, ureter).

In some embodiments, SCP are combined onto the scaffold in combination (e.g., as a co-culture or as separate layers of cells) with stem cells and/or cells of a soft tissue phenotype. The cells to be co-inoculated with the SCP will depend upon the tissue to be simulated. For example, SCP may be combined with the scaffold with epithelial cells (e.g., cells of oral mucosa, gastrointestinal tract, nasal epithelium, respiratory tract epithelium, vaginal epithelium, corneal epithelium), bone marrow cells, adipocytes, stem cells, keratinocytes, vascular endothelial cells (e.g., aortic endothelial cells, coronary artery endothelial cells, pulmonary artery endothelial cells, iliac artery endothelial cells, microvascular endothelial cells, umbilical artery endothelial cells, umbilical vein endothelial cells, and endothelial progenitors (e.g., CD34+, CD34+/CD117+ cells)), bladder urothelial cells, smooth muscle cells, gastrointestinal cells, esophageal cells, larynx cells, mucosal cells, myoblasts, myocytes, stromal cells, and other soft tissue cells or progenitor cells.

The three-dimensional scaffold of the invention may be used in skin grafting. Preferably, the scaffold is about 0.5 to about 3 millimeter thick and is in the form of a flat sheet. The scaffold is preferably combined with SCP. The scaffolds may be inoculated with at least one of stem cells, epithelial cells, dermal fibroblasts, melanocytes, and keratinocytes. In some embodiments, keratinocytes form a layer over the SCP combined scaffold. The scaffolds of the invention preferably comprise at least one of collagen, elastin, intercellular adhesion molecules, neural cell adhesion molecules, laminin, heparin binding growth factor, fibronectin, proteoglycan, tenascin, E-cahedrin, and fibrillin.

As another example, the three-dimensional scaffold may be used to generate muscle tissue. The scaffold is preferably seeded with SCP or SCP products. The scaffolds may be co-inoculated with at least one of stem cells, myocytes, and myoblasts.

The three-dimensional scaffold may be modified so that the growth of cells and the production of tissue thereon or therein is enhanced, or so that the risk of rejection of the implant is reduced. Thus, one or more biologically active compounds, including, but not limited to, antiapoptotic agents, anti-inflammatories, angiogenic factors, immunosuppressants or growth factors, may be added to the scaffold.

### Kits

The SCP can conveniently be employed as part of a kit, for example, for culture or in vivo administration. Accordingly, the invention provides a kit including the SCP and additional components, such as a matrix (e.g., a scaffold), hydrating agents (e.g., physiologically-compatible saline solutions, prepared cell culture media), cell culture substrates (e.g., culture dishes, plates, vials, etc.), cell culture media (whether in liquid or powdered form), antibiotic compounds, hormones, a bioactive factor, a second cell type, a differentiation-inducing agent, cell culture media, and the like. While the kit can include any such components, preferably it includes all ingredients necessary for its intended use. If desired, the kit also can include cells (typically cryopreserved), which can be seeded into the lattice as described herein.

In another aspect, the invention provides kits that utilize the SCP in various methods for augmentation, regeneration, and repair as described above. In some embodiments, the kits may include one or more cell populations, including at least SCP and a pharmaceutically acceptable carrier (liquid, semi-solid or solid). The kits also optionally may include a means of administering the SCP, for example by injection. The kits further may include instructions for use of the SCP. Kits prepared for field hospital use, such as for military use, may include full-procedure supplies including tissue scaffolds, surgical sutures, and the like, where the cells are to be used in conjunction with repair of acute injuries. Kits for assays and in vitro methods as described herein may contain one or more of (1) SCP, (2) reagents for practicing the in vitro method, (3) other cells or cell populations, as appropriate, and (4) instructions for conducting the in vitro method.

### EXAMPLES

### Example 1

### Derivation of Cells from Postpartum Tissues

In this study populations of cells from placental and umbilicus tissues were derived. Postpartum umbilicus and placenta were obtained upon birth of either a full term or pre-term pregnancy. Cells were harvested from 5 separate donors of umbilicus and placental tissue. Different methods of cell isolation were tested for their ability to yield cells with: 1) the potential to differentiate into cells with different phenotypes, or 2) the potential to provide critical trophic factors useful for other cells and tissues.

Methods & Materials

Umbilicus cell derivation. Umbilical cords were obtained from National Disease Research Interchange (NDRI, Philadelphia, Pa.). The tissues were obtained following normal deliveries. The cell isolation protocol was performed aseptically in a laminar flow hood. To remove blood and debris, the umbilicus was washed in phosphate buffered saline (PBS; Invitrogen, Carlsbad, Calif.) in the presence of antimycotic and antibiotic (100 Units/milliliter penicillin, 100 micrograms/milliliter streptomycin, 0.25 micrograms/milliliter amphotericin B) (Invitrogen Carlsbad, Calif.)). The tissues were then mechanically dissociated in 150 cm² tissue culture plates in the presence of 50 milliliters of medium (DMEM-Low glucose or DMEM-High glucose; Invitrogen) until the tissue was minced into a fine pulp. The chopped tissues were transferred to 50 milliliter conical tubes (approximately 5 grams of tissue per tube). The tissue was then digested in either DMEM-Low glucose medium or DMEM-High glucose medium, each containing antimycotic and antibiotic (100 Units/milliliter penicillin, 100 micrograms/milliliter streptomycin, 0.25 micrograms/milliliter amphotericin B (Invitrogen)) and digestion enzymes. In some experiments, an enzyme mixture of collagenase and dispase was used (“C:D;” collagenase (Sigma, St Louis, Mo.), 500 Units/milliliter; and dispase (Invitrogen), 50 Units/milliliter in DMEM-Low glucose medium). In other experiments a mixture of collagenase, dispase and hyaluronidase (“C:D:H”) was used (collagenase, 500 Units/milliliter; dispase, 50 Units/milliliter; and hyaluronidase (Sigma), 5 Units/milliliter, in DMEM-Low glucose). The conical tubes containing the tissue, medium and digestion enzymes were incubated at 37° C. in an orbital shaker (Environ, Brooklyn, N.Y.) at 225 rpm for 2 hrs.

After digestion, the tissues were centrifuged at 150×g for 5 minutes, and the supernatant was aspirated. The pellet was resuspended in 20 milliliters of Growth medium (DMEM-Low glucose (Invitrogen), 15 percent (v/v) fetal bovine serum (FBS; defined bovine serum; Lot#AND18475; Hyclone, Logan, Utah), 0.001% (v/v) 2-mercaptoethanol (Sigma), 100 Units/milliliter of penicillin, 100 microgram/milliliter streptomycin, 0.25 microgram/milliliter amphotericin B (Invitrogen, Carlsbad, Calif.). The cell suspension was filtered through a 70-micrometer nylon cell strainer (BD Biosciences). An additional 5 milliliter rinse comprising Growth medium was passed through the strainer. The cell suspension was then passed through a 40-micrometer nylon cell strainer (BD Biosciences) and chased with a rinse of an additional 5 milliliters of Growth medium.

The filtrate was resuspended in Growth medium (total volume 50 milliliters) and centrifuged at 150×g for 5 minutes. The supernatant was aspirated, and the cells were resuspended in 50 milliliters of fresh Growth medium. This process was repeated twice more.

Upon the final centrifugation supernatant was aspirated and the cell pellet was resuspended in 5 milliliters of fresh Growth medium. The number of viable cells was determined using Trypan Blue staining. Cells were then cultured under standard conditions.

The cells isolated from umbilicus were seeded at 5,000 cells/cm² onto gelatin-coated T-75 cm² flasks (Corning Inc., Corning, N.Y.) in Growth medium (DMEM-Low glucose (Invitrogen), 15 percent (v/v) defined bovine serum (Hyclone, Logan, Utah; Lot#AND18475), 0.001 percent (v/v) 2-mercaptoethanol (Sigma), 100 Units/milliliter penicillin, 100 micrograms/milliliter streptomycin, 0.25 micrograms/milliliter amphotericin B (Invitrogen)). After about 2-4 days, spent medium was aspirated from the flasks. Cells were washed with PBS three times to remove debris and blood-derived cells. Cells were then replenished with Growth medium and allowed to grow to confluence (about 10 days from passage 0 to passage 1). On subsequent passages (from passage 1 to 2, etc.), cells reached sub-confluence (75-85 percent confluence) in 4-5 days. For these subsequent passages, cells were seeded at 5000 cells/cm². Cells were grown in a humidified incubator with 5 percent carbon dioxide and 20 percent oxygen at 37° C.

Placental Cell Isolation. Placental tissue was obtained from NDRI (Philadelphia, Pa.). The tissues were from a pregnancy and were obtained at the time of a normal surgical delivery. Placental cells were isolated as described for umbilicus cell isolation.

The following example applies to the isolation of separate populations of maternal-derived and neonatal-derived cells from placental tissue.

The cell isolation protocol was performed aseptically in a laminar flow hood. The placental tissue was washed in phosphate buffered saline (PBS; Invitrogen, Carlsbad, Calif.) in the presence of antimycotic and antibiotic (100 Units/milliliter penicillin, 100 microgram/milliliter streptomycin, 0.25 microgram/milliliter amphotericin B; Invitrogen) to remove blood and debris. The placental tissue was then dissected into three sections: top-line (neonatal side or aspect), mid-line (mixed cell isolation neonatal and maternal or villous region), and bottom line (maternal side or aspect).

The separated sections were individually washed several times in PBS with antibiotic/antimycotic to further remove blood and debris. Each section was then mechanically dissociated in 150 cm² tissue culture plates in the presence of 50 milliliters of DMEM-Low glucose (Invitrogen) to a fine pulp. The pulp was transferred to 50 milliliter conical tubes. Each tube contained approximately 5 grams of tissue. The tissue was digested in either DMEM-Low glucose or DMEM-High glucose medium containing antimycotic and antibiotic (100 Units/milliliter penicillin, 100 micrograms/milliliter streptomycin, 0.25 micrograms/milliliter amphotericin B (Invitrogen)) of PBS and digestion enzymes. In some experiments an enzyme mixture of collagenase and dispase (“C:D”) was used containing collagenase (Sigma, St Louis, Mo.) at 500 Units/milliliter and dispase (Invitrogen) at 50 Units/milliliter in DMEM-Low glucose medium. In other experiments a mixture of collagenase, dispase, and hyaluronidase (C:D:H) was used (collagenase, 500 Units/milliliter; dispase, 50 Units/milliliter; and hyaluronidase (Sigma), 5 Units/milliliter in DMEM-Low glucose). The conical tubes containing the tissue, medium, and digestion enzymes were incubated for 2 h at 37° C. in an orbital shaker (Environ, Brooklyn, N.Y.) at 225 rpm.

After digestion, the tissues were centrifuged at 150×g for 5 minutes, the resultant supernatant was aspirated off. The pellet was resuspended in 20 milliliter of Growth medium (DMEM-Low glucose (Invitrogen), 15% (v/v) fetal bovine serum (FBS; defined bovine serum; Lot#AND18475; Hyclone, Logan, Utah), 0.001% (v/v) 2-mercaptoethanol (Sigma, St. Louis, Mo.), antibiotic/antimycotic (100 Units/milliliter penicillin, 100 microgram/milliliter streptomycin, 0.25 microgram/milliliter amphotericin B; Invitrogen)). The cell suspension was filtered through a 70 micrometer nylon cell strainer (BD Biosciences), chased by a rinse with an additional 5 milliliters of Growth medium. The total cell suspension was passed through a 40 micrometer nylon cell strainer (BD Biosciences) followed with an additional 5 milliliters of Growth medium as a rinse.

The filtrate was resuspended in Growth medium (total volume 50 milliliters) and centrifuged at 150×g for 5 minutes. The supernatant was aspirated and the cell pellet was resuspended in 50 milliliters of fresh Growth medium. This process was repeated twice more. After the final centrifugation, supernatant was aspirated and the cell pellet was resuspended in 5 milliliters of fresh Growth medium. A cell count was determined using the Trypan Blue Exclusion test. Cells were then cultured at standard conditions.

LIBERASE (Boehringer Mannheim Corp., Indianapolis, Ind.) Cell Isolation. Cells were isolated from umbilicus in DMEM-Low glucose medium with LIBERASE (Boehringer Mannheim Corp., Indianapolis, Ind.) (2.5 milligrams per milliliter, Blendzyme 3; Roche Applied Sciences, Indianapolis, Ind.) and hyaluronidase (5 Units/milliliter, Sigma). Digestion of the tissue and isolation of the cells was as described for other protease digestions above using a LIBERASE (Boehringer Mannheim Corp., Indianapolis, Ind.)/hyaluronidase mixture in place of the C:D or C:D:H enzyme mixture. Tissue digestion with LIBERASE (Boehringer Mannheim Corp., Indianapolis, Ind.) resulted in the isolation of cell populations from postpartum tissues that expanded readily.

Cell isolation using other enzyme combinations. Procedures were compared for isolating cells from the umbilicus using differing enzyme combinations. Enzymes compared for digestion included: i) collagenase; ii) dispase; iii) hyaluronidase; iv) collagenase:dispase mixture (C;D); v) collagenase:hyaluronidase mixture (C:H); vi) dispase:hyaluronidase mixture (D:H); and vii) collagenase:dispase:hyaluronidase mixture (C:D:H). Differences in cell isolation utilizing these different enzyme digestion conditions were observed (Table 1-1).

Isolation of cells from residual blood in the cords. Attempts were made to isolate pools of cells from umbilicus by different approaches. In one instance umbilical cord was sliced and washed with Growth medium to dislodge the blood clots and gelatinous material. The mixture of blood, gelatinous material, and Growth medium was collected and centrifuged at 150×g. The pellet was resuspended and seeded onto gelatin-coated flasks in Growth medium. From these experiments a cell population was isolated that readily expanded.

Isolation of cells from Cord Blood. Cells have also been isolated from cord blood samples attained from NDRI. The isolation protocol used here was that of International Patent Application WO02/29971 by Ho et al. Samples (50 milliliters and 10.5 milliliters, respectively) of umbilical cord blood (NDRI, Philadelphia Pa.) were mixed with lysis buffer (filter-sterilized 155 millimolar ammonium chloride, 10 millimolar potassium bicarbonate, 0.1 millimolar EDTA buffered to pH 7.2 (all components from Sigma, St. Louis, Mo.)). Cells were lysed at a ratio of 1:20 cord blood to lysis buffer. The resulting cell suspension was vortexed for 5 seconds, and incubated for 2 minutes at ambient temperature. The lysate was centrifuged (10 minutes at 200×g). The cell pellet was resuspended in complete minimal essential medium (Gibco, Carlsbad Calif.) containing 10 percent fetal bovine serum (Hyclone, Logan Utah), 4 millimolar glutamine (Mediatech Herndon, Va.), 100 Units penicillin per 100 milliliters and 100 micrograms streptomycin per 100 milliliters (Gibco, Carlsbad, Calif.). The resuspended cells were centrifuged (10 minutes at 200×g), the supernatant was aspirated, and the cell pellet was washed in complete medium. Cells were seeded directly into T75 flasks (Corning, N.Y.), T75 laminin-coated flasks, or T175 fibronectin-coated flasks (both Becton Dickinson, Bedford, Mass.).

Isolation of postpartum-derived cells using different enzyme combinations and growth conditions. To determine whether cell populations can be isolated under different conditions and expanded under a variety of conditions immediately after isolation, cells were digested in Growth medium with or without 0.001 percent (v/v) 2-mercaptoethanol (Sigma, St. Louis, Mo.), using the enzyme combination of C:D:H, according to the procedures provided above. Placenta-derived cells so isolated were seeded under a variety of conditions. All cells were grown in the presence of penicillin/streptomycin.

In all conditions, cells attached and expanded well between passage 0 and 1 (Table 1-2). Cells in conditions 5 to 8 and 13 to 16 were demonstrated to proliferate well up to 4 passages after seeding at which point they were cryopreserved. All cells were banked.

Results

Cell isolation using different enzyme combinations. The combination of C:D:H provided the best cell yield following isolation and generated cells which expanded for many more generations in culture than the other conditions (Table 1-1). An expandable cell population was not attained using collagenase or hyaluronidase alone. No attempt was made to determine if this result is specific to the collagen that was tested.

Isolation of postpartum-derived cells using different enzyme combinations and growth conditions. Cells attached and expanded well between passage 0 and 1 under all conditions tested for enzyme digestion and growth (Table 1-2). Cells in experimental conditions 5-8 and 13-16 proliferated well up to 4 passages after seeding, at which point they were cryopreserved. All cells were banked.

Isolation of cells from residual blood in the cords. Nucleated cells attached and grew rapidly. These cells were analyzed by flow cytometry and were similar to cells obtained by enzyme digestion.

Isolation of cells from Cord Blood. The preparations contained red blood cells and platelets. No nucleated cells attached and divided during the first 3 weeks. The medium was changed 3 weeks after seeding and no cells were observed to attach and grow.

Summary. Populations of cells can be isolated from umbilical cord and placental tissue most efficiently using the enzyme combination collagenase (a matrix metalloprotease), dispase (neutral protease), and hyaluronidase (a mucolytic enzyme which breaks down hyaluronic acid). LIBERASE (Boehringer Mannheim Corp., Indianapolis, Ind.), which is a Blendzyme, may also be used. In the present study Blendzyme 3 which is collagenase (4 Wunsch units/g) and thermolysin (1714 casein Units/g) was also used together with hyaluronidase to isolate cells. These cells expand readily over many passages when cultured in Growth medium on gelatin-coated plastic.

Postpartum-derived cells were isolated from residual blood in the cords but not from cord blood. The presence of cells in blood clots washed from the tissue that adhere and grow under the conditions used may be due to cells being released during the dissection process.

TABLE 1-1
Isolation of cells from umbilical cord tissue using varying
enzyme combinations

| Enzyme Digest | Cells Isolated | Cell Expansion |
|---|---|---|
| Collagenase | X | X |
| Dispase | + (>10 h) | + |
| Hyaluronidase | X | X |
| Collagenase:Dispase | ++ (<3 h) | ++ |
| Collagenase:Hyaluronidase | ++ (<3 h) | + |
| Dispase:Hyaluronidase | + (>10 h) | + |
| Collagenase:Dispase:Hyaluronidase | +++ (<3 h) | +++ |

Key:
+ = good,
++ = very good,
+++ = excellent,
X = no success

TABLE 1-2
Isolation and culture expansion of postpartum-derived cells
under varying conditions:

| Condition | Medium | 15% FBS | BME | Gelatin | 20% O2 | Growth Factors |
|---|---|---|---|---|---|---|
| 1 | DMEM-Lg | Y | Y | Y | Y | N |
| 2 | DMEM-Lg | Y | Y | Y | N (5%) | N |
| 3 | DMEM-Lg | Y | Y | N | Y | N |
| 4 | DMEM-Lg | Y | Y | N | N (5%) | N |
| 5 | DMEM-Lg | N (2%) | Y | N (Laminin) | Y | EGF/FGF (20 ng/mL) |
| 6 | DMEM-Lg | N (2%) | Y | N (Laminin) | N (5%) | EGF/FGF (20 ng/mL) |
| 7 | DMEM-Lg | N (2%) | Y | N | Y | PDGF/VEGF |
| | | | | (Fibronectin) | | |
| 8 | DMEM-Lg | N (2%) | Y | N | N (5%) | PDGF/VEGF |
| | | | | (Fibronectin) | | |
| 9 | DMEM-Lg | Y | N | Y | Y | N |
| 10 | DMEM-Lg | Y | N | Y | N (5%) | N |
| 11 | DMEM-Lg | Y | N | N | Y | N |
| 12 | DMEM-Lg | Y | N | N | N (5%) | N |
| 13 | DMEM-Lg | N (2%) | N | N (Laminin) | Y | EGF/FGF (20 ng/mL) |
| 14 | DMEM-Lg | N (2%) | N | N (Laminin) | N (5%) | EGF/FGF (20 ng/mL) |
| 15 | DMEM-Lg | N (2%) | N | N | Y | PDGF/VEGF |
| | | | | (Fibronectin) | | |
| 16 | DMEM-Lg | N (2%) | N | N | N (5%) | PDGF/VEGF |
| | | | | (Fibronectin) | | |

### REFERENCE

1. Ho et al., WO2003/025149 A2, CELL POPULATIONS WHICH CO-EXPRESS CD49C AND CD90, NEURONYX, INC., Application No. PCT/US02/29971, Filed 2002 Aug. 20, A2 Published 2003 Mar. 27, A3 Published 2003 Dec. 18.

### Example 2

### Production of Lyophilized Stem Cell Lysate

The purpose of this study was to provide methods for the production of lyophilized stem cell lysate. The method consistently allowed the harvest of proteins from lysed stem cells. The amount of total protein (57.53+/−38.69 picograms per cell) correlates to the harvest density of the hUTC (R-Sq (adj)=71.5%). hMSC lysate yielded 107.29 picograms of total protein per cell. The growth factor bFGF was present in six separate production lots of lyophilized hUTC lysate averaging 3.09+/−1.06 picograms per microgram of total protein. SDS-PAGE analysis of hUTC lysate showed the banding pattern of protein was consistent between separate production lots, pre- and post-lyophilization, and lyophilization into a synthetic biomaterial. The current method allowed reproducible production of lyophilized material containing growth factors for application in tissue regeneration.

Methods & Materials

Cell Growth and Harvest. hUTCs were seeded at 5,000 cells per cm² in gelatin-coated flasks with growth media (Dulbecco's Modified Eagles Media (DMEM)-low glucose, 15% fetal bovine serum (FBS), penicillin/streptomycin (P/S), Betamercaptoethanol (BME) and expanded for 3 to 4 days (25,000 cells per cm² target harvest density). Cells were harvested with trypsin, collected, and centrifuged at 300 rcf for 5 minutes. The trypsin/media was removed by aspiration and cells were washed three times with phosphate buffered saline (PBS).

Human Mesenchymal Stem Cells obtained from Cambrex (Walkersville, Md. cat no 1560) were seeded at 5,000 cells per cm squared in T-flasks with growth media (cat. no. PT-3001). The cells were expanded for 3 to 4 days and at 70% confluency, harvested with trypsin, collected, and centrifuged at 300 rcf for 5 minutes. The trypsin/media was removed by aspiration and cells were washed three times with phosphate buffered saline (PBS). Cells were harvested at passage 6.

Cell Wash and Aliquoting. After washing, the cells were re-suspended at 1.0E+07 cell/ml in PBS and delivered as 1 ml aliquots into 1.5 ml sterile siliconized micro-centrifuge tubes. The cells were centrifuged at 300 rcf for 5 minutes and the PBS was removed by aspiration. Tubes containing cell pellets were optionally stored at −80° C.

Cell Lysis. Tubes containing cell pellets were immersed in liquid nitrogen (LN2) for 60 seconds. The tubes were then removed from LN2 and immediately immersed in a 37° C. water bath for 60 seconds or until thawed (3 minute maximum incubation time). This process was repeated two additional times.

Centrifugation and Lysate Harvest. The freeze-thawed samples were centrifuged for 10 minutes at 13,000 rcf at 4° C. and placed on ice. The supernatant fluid from each tube was removed by pipette and transferred to a single sterile siliconized 1.5 ml tube. This process was repeated until no additional supernatant fluid could be recovered.

Fluid Volume Measurement. To approximate supernatant fluid volume, the 1.5 ml tube containing recovered supernatant fluid was weighed on a balance previously tared with an empty 1.5 ml micro-centrifuge tube (1 milligram=˜1 microliter).

Protein Assay. To determine total protein content, 10 microliters of lysate supernatant fluid was diluted into 990 microliters PBS, and the dilution was analyzed by Bradford assay (standard range 1.25-25 micrograms). This value was used to calculate the total protein per cell, the main metric used to ensure the consistency of the process.

Lysate Lyophilization. Multiple 1.5 milliliter sterile labeled cryovials were loaded into a sterile heat transfer block. Aliquots of lysate supernatant fluid at defined total protein concentration were loaded into the cryovials. The heat block containing uncapped cryovials was aseptically loaded into an autoclaved pouch with tube openings facing the paper side of the pouch. The pouch was sealed before removal from the laminar flow hood. The pouch was loaded into the lyophilizer.

Pre-cut materials (i.e., 90/10 PGA/PLA non-woven) were aseptically placed into the wells of 24- or 48-well sterile, ultra low cluster cell culture dishes (Corning Inc., Corning N.Y.). Lysate supernatant fluid was delivered at a defined total protein concentration onto the material. For example, a material measuring 6 mm in diameter and 0.5 mm in thickness received 2 microliters of a 15 microgram/microliter total protein solution to create a 30 microgram lysate protein material. The lid of the dish was replaced and secured with tape. The dish with materials was loaded into the lyophilizer

Test materials with applied lysate were loaded into a FTS Systems Dura-Stop MP Stoppering Tray Dryer and lyophilized using the following ramping program. All steps had a ramping rate of 2.5° C./minute and a 100-mT vacuum.

TABLE 2-1
Ramping program utilized for the lyophilization of hUTC
lysate

| | | Hold Time |
|---|---|---|
| Step | Shelf Temp (° C.) | (minutes) |
| a | −40 | 180 |
| b | −25 | 2160 |
| c | −15 | 180 |
| d | −5 | 180 |
| e | 5 | 120 |
| f | 20 | 120 |
| g | −20 | 60 |

bFGF Enyme Linked Immunosorbent Assay (ELISA) Analysis. Vials from six separate production lots of lyophilized lysate powder were reconstituted in PBS and analyzed for total protein content by Bradford assay. The samples were then further diluted to achieve a 20 microgram/milliliter solution. Solutions further serially diluted in PBS and analyzed by ELISA using a Quantikine human bFGF kit (R&D Systems cat. no. DFB50).

SDS-PAGE. Polyacrylamide Gel Electrophoresis (PAGE) was conducted under denaturing conditions using sodium dodecylsulfate (SDS) using the NOVEX mini gel system (Invitrogen, Carlsbad, Calif.). Samples were prepared with the NOVEX Tris-Glycine SDS Sample Buffer (Invitrogen, Carlsbad, Calif.) using the manufacturer's suggested protocol. Samples for analysis included: a) hUTC lysate prior to lyophilization, b) hUTC lysate lyophilized in vials, and c) hUTC lysate lyophilized onto 90/10 PGA/PLA non-woven materials. The samples were loaded onto a NOVEX Pre-Cast Tris-Glycine 4-20% Stacking Mini Gel and run in the XCell Sure Lock Mini-Cell with NOVEX Tris-Glycine Running Buffer for the manufacturer suggested time and voltage (Invitrogen, Carlsbad, Calif.). Gels were stained with SIMPLYBLUE Safe Stain and dried using the DRYEASE Mini-Gel Drying System (Invitrogen, Carlsbad, Calif.) according to the manufacturer's instructions.

Results

Lyophilized Lysate Production Summary

TABLE 2-2
Metrics summary from multiple production lots of hUTC
lysate

| | | | | | | Total |
|---|---|---|---|---|---|---|
| | | Total T225 | | | | protein |
| | | culture | Harvest | Total ul | | (ug)/total |
| | Total cells | flasks | density | lysate | Total Protein | lysate |
| Lot | harvested | used | (cells/cm²) | fluid | (ug) | fluid (ul) |
| L011905A | 2.55E+08 | 30 | 38000 | 875 | 27063.8 | 30.93 |
| L011905B | 5.42E+07 | 8 | 31000 | 117 | 3068.9 | 26.23 |
| L011905C | 1.84E+08 | 26 | 32000 | 597 | 18614.5 | 31.18 |
| L030705 | 1.05E+08 | 20 | 23000 | 389 | 7869.5 | 20.23 |
| L033105 | 1.05E+08 | 25 | 18700 | 257 | 6296.5 | 24.5 |
| L040405 | 5.95E+08 | 165 | 16000 | 1394 | 16072.8 | 11.53 |
| L042205 | 2.64E+08 | 100 | 11700 | 528 | 7920 | 15 |
| L051305 | 1.70E+08 | 101 | 7500 | 609 | 2192.4 | 3.6 |
| L052505 | 4.00E+07 | 8 | 22222 | 147 | 529 | 3.6 |
| L061305 | 3.60E+08 | 39 | 40600 | 934 | 46700 | 50 |
| L062405 | 3.20E+08 | 60 | 23800 | 424 | 17000 | 40 |
| L071305 | 4.60E+08 | 100 | 20400 | 922 | 10879 | 11.8 |
| Totals | 2.91E+09 | — | — | 7.19E+03 | 1.64E+05 | — |

Total Protein per Cell/Harvest Density Correlation. The total protein content of recovered lysate supernatant fluid prior to lyophilization is a function of the cell density at time of harvest (R-Sq (adj)=71.5%).

TABLE 2-3
Correlation between total protein per hUTC and cell
density at time of harvest

| | | Harvest | | Protein per |
|---|---|---|---|---|
| | | density | Total protein | cell |
| Lot | Total Cells | (cells/cm²) | (picograms) | (picograms) |
| L011905A | 2.55E+08 | 38000 | 2.71E+10 | 106.13 |
| L011905B | 5.42E+07 | 31000 | 3.07E+09 | 56.62 |
| L011905C | 1.84E+08 | 32000 | 1.86E+10 | 101.17 |
| L030705 | 1.05E+08 | 23000 | 7.87E+09 | 74.95 |
| L033105 | 1.05E+08 | 18700 | 6.30E+09 | 59.97 |
| L040405 | 5.95E+08 | 16000 | 1.61E+10 | 27.01 |
| L042205 | 2.64E+08 | 11700 | 7.92E+09 | 30.00 |
| L051305 | 1.70E+08 | 7500 | 2.19E+09 | 12.90 |
| L052505 | 4.00E+07 | 22222 | 5.29E+08 | 13.23 |
| L061305 | 3.60E+08 | 40600 | 4.67E+10 | 129.72 |
| L062405 | 3.20E+08 | 23800 | 1.70E+10 | 53.00 |
| L071305 | 4.60E+08 | 21000 | 1.18E+10 | 25.65 |
| Avg. | — | — | — | 57.53 |
| Std. Dev. | — | — | — | 38.69 |

TABLE 2-3
Total protein per hMSC and cell density at time of harvest

| | | Harvest | | Protein per |
|---|---|---|---|---|
| | | density | Total protein | cell |
| Lot | Total Cells | (cells/cm²) | (picograms) | (picograms) |
| LM041906 | 1.7E+07 | 6868 | 1.8E+9 | 107.29 |

bFGF ELISA Analysis hUTC lysate

TABLE 2-4
Summary of bFGF (picograms) per given quantity of total
lysate protein as measured by ELISA assay

| | 2.5 | | 10 | 20 |
|---|---|---|---|---|
| | micrograms | 5 micrograms | micrograms | micrograms |
| | total protein | total protein | total protein | total protein |
| L040405 | 16.3 | 29.48 | 64.07 | 129.14 |
| L042205 | 16.61 | 26.399 | 54.944 | 116.521 |
| L051305 | 11.08 | 17.01 | 34.6 | 79.02 |
| L052505 | 14.277 | 22.105 | 47.28 | 110.39 |
| L061305 | 10.26 | 15.13 | 28.92 | 61.936 |
| L062405 | 15.5 | 24.5 | 51.89 | 112.951 |

TABLE 2-5
Regression analysis of bFGF content of PBS reconstituted
and serially diluted lyophilized hUTC lysate from six separate
production lots

| | | | | Picograms |
|---|---|---|---|---|
| | | | | bFGF per |
| | | | | microgram |
| Lot | Slope | y-intercept | R squared | total protein |
| L040405 | 37.31 | −33.53 | 0.91 | 3.78 |
| L042205 | 32.82 | −28.45 | 0.89 | 4.37 |
| L051305 | 22.14 | −19.95 | 0.87 | 2.19 |
| L052505 | 31.35 | −29.86 | 0.86 | 1.49 |
| L061305 | 16.88 | −13.43 | 0.88 | 3.45 |
| L062405 | 31.97 | −28.72 | 0.88 | 3.25 |
| Average | 28.75 | −25.66 | — | 3.09 |
| Std. Dev. | 7.64 | 7.47 | — | 1.06 |

Calculated concentration of bFGF per lyophilized hUTC lysate total protein yielded the following equation:

bFGF (picograms/milliliter)=(28.745) total protein (micrograms/milliliter)−25.656.

### Equation slope and Y-intercept are derived from the average slope and Y-intercept values obtained from regression analysis of six production lots.

SDS-PAGE analysis of hUTC lysate. Banding pattern of protein is consistent between separate production lots, pre- and post-lyophilization, and lyophilization onto a synthetic biomaterial.

Summary. The method presented here consistently allowed for the harvest of protein from lysed, centrifuged hUTCs. The amount of total protein—57.53+/38.69 picograms per cell—correlates to the harvest density of the cells (R-Sq (adj)=71.5%). The growth factor bFGF was present in six separate production lots of lyophilized hUTC lysate averaging 3.09±1.06 picograms per micrograms of total protein. SDS-PAGE analysis of umbilicus derived cell lysate showed that the banding pattern of protein was consistent between separate production lots, pre- and post-lyophilization, and following lyophilization onto a synthetic biomaterial. This method allows reproducible production of lyophilized material containing growth factors for application in tissue regeneration.

### Example 3

### Analyses of Factors Present in the Cell Lysate as Determined by Multiplex ELISA

Methods & Materials

Preparation of Cell Lysate. Approximately 25 million human umbilicus-derived cells (hUTCs) at passage 11 were seeded into gelatin-coated T225 flasks. Because of the number of cells that were necessary to complete the study, the flasks were split, for trypsinization, into two sets which were combined to prepare the cell lysate. The cells ranged from approximately 70-95% confluent. Flasks were trypsinized with 0.05% trypsin/EDTA for 5 minutes until the cells began lifting from the dish. The trypsinization process was inactivated using 15% serum containing Dulbecco's Modified Eagle's growth media. Cells were pelleted in growth media and then resuspended in a total volume of 40 milliliters of PBS. The cells were washed three times in PBS to remove residual FBS from the growth media. This was done by centrifuging the cells for 5 minutes at 1.5 RPM and then resuspending the cells in 40 milliliters of PBS until the three washes were complete.

In order to facilitate the freeze-thaw procedure, the cells were equally divided into two tubes with PBS for the freeze/thaw procedure. The lysates were prepared by repeated freeze/thaw cycles. To freeze the cells, the tubes were placed in a slurry of dry ice and isopropanol for 10 minutes. After 10 minutes, the tubes were placed in a 37° C. water bath for 10 minutes.

The cell suspensions were transferred to ten sterile siliconized microcentrifuge tubes, to prevent protein adsorption, and centrifuged at 13,000×g for 10 minutes at 4° C. to separate the cell membranes from the cytosolic components. The tubes (cell pellet) were then placed on ice and the supernatant was very gently mixed by tapping the centrifuge tube to ensure uniformity. The supernatant was transferred to new siliconized tubes and placed on ice.

SEARCHLIGHT Multiplexed ELISA assay. Chemokines, BDNF and angiogenic factors were measured using SEARCHLIGHT Proteome Arrays (Pierce Biotechnology Inc.). The proteome arrays are multiplexed sandwich ELISAs for the quantitative measurement of two to 16 proteins per well. The arrays are produced by spotting a 2×2, 3×3, or 4×4 pattern of four to 16 different capture antibodies into each well of a 96-well plate. Following a typical sandwich ELISA procedure, the entire plate is imaged to capture chemiluminescent signal generated at each spot within each well of the plate. The amount of signal generated in each spot is proportional to the amount of target protein in the original standard or sample.

Results

TABLE 3-1
SEARCHLIGHT Multiplexed ELISA results. Average for
duplicate adjusted for dilution.

| ANG2 | HGF | HBEGF | KGF | | PDGFbb | VEGF | |
|---|---|---|---|---|---|---|---|
| (pg/ml) | (pg/ml) | (pg/ml) | (pg/ml) | FGF (pg/ml) | (pg/ml) | (pg/ml) | IL6 (pg/ml) |
| <41.2 | 64500.0 | 68.0 | 260.8 | 167500.0 | 4.8 | 76.6 | 258.8 |

| IL8 | MCP1 | TGFa | TIMP1 | TIMP2 | HGH | BDNF |
|---|---|---|---|---|---|---|
| (pg/ml) | (pg/ml) | (pg/ml) | (pg/ml) | (pg/ml) | (pg/ml) | (pg/ml) |
| 14700.0 | 197.4 | 208.0 | 6865.0 | 25460.0 | 236.0 | 1115.2 |

Summary. hUTC lysate contains significant levels of beneficial factors including pro-angiogenic as well as factors that can stimulate cell proliferation and extracellular matrix production (KGF, PDGF-BB, HGF, TGFa) and neurotrophic factors (BDNF, IL-6). These factors might have beneficial effects on local environment by inducing cell proliferation, differentiation and survival. In addition, pro-angiogenic factors might induce new blood vessel formation in the wound environment and stimulate extracellular matrix formation. Furthermore, the high level of TIMPs might be extremely beneficial in the chronic wound environment, since chronic wounds are known to be associated with high levels of MMPs, known to mediate extracellular matrix degradation.

### Example 4

### Effect of Collagen/ORC Material Containing Cell Lysate on Mouse NIH/3T3 Fibroblast Proliferation in a Co-Culture Transwell System

Introduction

It is well known that multiple processes involving the sequential expression of various proteins are necessary for optimal tissue repair and remolding. Based on this concept, optimal tissue cannot be achieved by the administration of a single bioactive factor. Because of the complexity of tissue restoration processes, various factors such as growth factors, and cytokines involved in tissue restoration may be required for optimal repair.

Stem Cell Products contain various trophic factors involved in tissue regeneration. The application of the cell lysate to biomaterials followed by lyophilization, produces a device suitable for tissue engineering and regenerative medicine applications.

The current work evaluated the ability of lysate obtained from hUTC, and hMSC lyophilized onto a biomaterial to increase NIH/3T3 fibroblast proliferation when co-cultured in a transwell system. Collagen/ORC containing lyophilized cell lysate was placed in the upper portion of a transwell system and co-cultured with NIH/3T3 fibroblasts plated at low density in the lower portion of the system. After three days the cells were harvested and counted and the transwells containing materials were transferred to new transwell systems and co-cultured with NIH/3T3 fibroblasts plated at low density in the lower portion of the system. After an additional two days (five days total material time in culture), the cells were harvested and counted.

Materials and Methods

Cell Growth, Harvest, and Lysate Production hUTC lot number 120204 were seeded at 5,000 cells per cm squared in gelatin-coated flasks with growth media Dulbecco's Modified Eagles Media (DMEM)-low glucose, 15% fetal bovine serum (FBS), penicillin/streptomycin (P/S), betamercaptoethanol (BME) and expanded for 3 to 4 days (25,000 cells per cm squared target harvest density). Cells, at 70% confluency, were harvested with trypsin, collected, and centrifuged at 300 rcf for 5 minutes. The trypsin/media was removed by aspiration and cells were washed three times with phosphate buffered saline (PBS). Cells were harvested at passage 10.

Human Mesenchymal Stem Cells obtained from Cambrex (Walkersville, Md. cat no 1560) were seeded at 5,000 cells per cm squared in T-flasks with growth media (cat. no. PT-3001). The cells were expanded for 3 to 4 days and at 70% confluency, harvested with trypsin, collected, and centrifuged at 300 rcf for 5 minutes. The trypsin/media was removed by aspiration and cells were washed three times with phosphate buffered saline (PBS). Cells were harvested at passage 6.

Cell Wash and Aliquoting After washing, the cells were re-suspended at 1.0E+07 cell/ml in PBS and delivered as 1 ml aliquots into 1.5 ml sterile siliconized micro-centrifuge tubes. The cells were centrifuged at 300 rcf for 5 minutes and the PBS was removed by aspiration. Tubes containing cell pellets were stored at −80° C.

Cell Lysis Tubes containing cell pellet were immersed into liquid nitrogen (LN₂) for 60 seconds. The tubes were then remove from LN₂ and immediately immersed in a 37° C. water bath for 60 seconds or until thawed (3 minute maximum incubation time). This process was repeated two additional times (Cell SOP #15 v 1—Cell Lysate Production and Loading on Scaffold).

Centrifugation and Lysate Harvest The freeze-thawed samples were centrifuged for 10 minutes at 13,000 rcf at 4° C. and placed on ice. The supernatant fluid from each tube was removed by pipette and transferred to a single sterile siliconized 1.5 ml tube. This process was repeated until no additional supernatant fluid could be recovered. (Cell SOP #15 v 1—Cell Lysate Production and Loading on Scaffold).

Fluid Volume Measurement To approximate supernatant fluid volume, the 1.5 ml tube containing recovered supernatant fluid was weighed on a balance previously tarred with an empty 1.5 ml micro-centrifuge tube (1 mg=˜1 μl).

Protein Assay To determine total protein content, 10 μl of lysate supernatant fluid was diluted into 990 μl PBS and the dilution was analyzed by Bradford assay (standard range 1.25-25 μg). This value was used to calculate the total protein per cell, the main metric used to ensure the consistency of the process.

Lysate Application and Lyophilization Collagen/ORC pre-cut to 3 mm in diameter with a dermal biopsy punch were aseptically placed into the wells of 24 well sterile, ultra low cluster cell culture dishes (Corning Inc., Corning N.Y.). The supernatant fluid was applied to the material as 120 μg protein aliquots. The dish with materials was loaded into the lyophilizer

Lyophilization Test materials with applied lysate were loaded into a FTS Systems Dura-Stop MP Stoppering Tray Dryer and lyophilized using the following ramping program. All steps had a ramping rate of 2.5° C./minute and a 100-mT vacuum.

TABLE 1
Ramping program utilized for the lyophilization of cell lysate

| Step | Shelf Temp (° C.) | Hold Time (minutes) |
|---|---|---|
| a | −40 | 180 |
| b | −25 | 2160 |
| c | −15 | 180 |
| d | −5 | 180 |
| e | 5 | 120 |
| f | 20 | 120 |
| g | −20 | 60 |

Proliferation Target Cells NIH/3T3 fibroblasts (ATCC CRL-1658) were expanded in growth media (DMEM high glucose with 10% neonatal calf serum and penicillin/streptomycin).

All treatments had an n of 8.

10% NCS (empty transwell)

1% NCS (empty transwell)

Collagen/ORC in 1% NCS

Collagen/ORC containing hUTC lysate (120 μg) in 1% NCS

Collagen/ORC containing hMSC lysate (120 μg) in 1% NCS

Transwell Assay The NIH/3T3 fibroblasts were plated into the lower portion of a 96 well transwell plate (Corning cat. no. 3381) at 2,500 cells per cm squared and cultured overnight. The media was removed by aspiration and the appropriate media (150 μl per well, 50 μl per transwell), transwells, and treatments were added. On day 3, transwells containing materials were removed and transferred to new 96 well plates that were seeded with NIH/3T3 cells the prior day.

Cell Harvest and Analysis Cells in transwells were harvested by trypsinization and counted using a Guava 96 instrument and Guava ViaCount Flex reagents as per manufacturers instructions. (Guava Technologies, Hayward Calif.)

Statistical Analysis Data is represented as mean viable cells +/− the standard deviation. Statistical analysis performed using Microsoft Excel software.

Results

TABLE 4-2
Cells per well (96 well plate) after three days transwell co-
culture with treatment as calculated by Guava 96 instrument

| | Avg. Cells | |
|---|---|---|
| Material | per Well | Std Dev |
| 10% NSC | 16,816 | 2,700 |
| 1% NCS | 1,863 | 366 |
| Collagen/ORC + 120 ug hUTC Lysate | 2,812 | 802 |
| Collagen/ORC + 120 ug MSC Lysate | 3,212 | 1,237 |
| Collagen/ORC | 1,940 | 508 |

TABLE 4-3
Cells per well (96 well plate) after two days transwell co-
culture with transferred treatment (total five days in study)
as calculated by Guava 96 instrument.
(“\” indicates wells with no data was obtained).

| | Avg. Cells | |
|---|---|---|
| Material | per Well | Std Dev |
| 10% NSC | 13,796 | 2,247 |
| 1% NCS | 676 | 496 |
| Collagen/ORC + 120 ug hUTC Lysate | 5,756 | 738 |
| Collagen/ORC + 120 ug MSC Lysate | 2,290 | 891 |
| Collagen/ORC | 1,875 | 658 |

### CONCLUSION

At day three, a significant increase in proliferation (t-test, p=0.02) of NIH/3T3 fibroblasts co-cultured with collagen/ORC containing 120 ug hUTC lysate vs. collagen/ORC alone was observed. Also a significant increase in proliferation (t-test, p=0.01) of NIH/3T3 fibroblasts co-cultured for three days with collagen/ORC containing 120 ug hMSC lysate vs. collagen/ORC alone was observed.

At day five in culture, a significant increase in proliferation of NIH/3T3 fibroblasts co-cultured with collagen/ORC containing 120 ug hUTC lysate vs. collagen/ORC alone (t-test, p=1.5E−07) and vs. collagen/ORC containing 120 ug hMSC lysate (t-test, p=1.6E−06) was observed.

These results demonstrate the biological activity of the hUTC lysate or hMSC lysate lyophilized onto biomaterial scaffolds and tested in a transwell system.

### Example 5

### A 14-Day Evaluation of Proprietary Constructs Containing Post-Partum Cell Lysate on Wound Healing in db/db Mice

SCP lysate has been evaluated in several in vivo models previously. Two acute models have been used, a rat subcutaneous implant model and a full-thickness excisional swine model. These studies demonstrated that cell lysate has a good biocompatibility profile, yields increased extracellular matrix formation in the rat subcutaneous implant model (Examples 18 and 19) and results in increased extracellular matrix deposition at early timepoints in the pig with a concomitant increase in inflammation which is not present at day 14 (Examples 21 and 22). Additionally, SCP lysate has been evaluated in two delayed healing models, an ischemic rat model (Example 23) and in a full-thickness excisional wound model in db/db mice (Example 24). In the ischemic rat model, a greater than two-fold increase in angiogenesis was observed in wounds treated with biomaterials containing SCP lysate compared to saline control. In the previous db/db model, although wound closure was not achieved due to the nonresorbable scaffold material bridging the wound open, enhanced granulation tissue formation was seen in the cell lysate groups.

The purpose of this study was to evaluate the biological effect of hUTC lysate lyophilized onto and released from a natural scaffold consisting of collagen/ORC in a recognized model of delayed healing, the db/db mouse wound healing model. The primary endpoint considered in this evaluation was the effect of this material on the increase in the healing rate (time to complete wound closure) in this impaired model since this is the key requirement set forth from the FDA Guidance for Industry for Development of Products for Treatment of Cutaneous Ulcers. Qualitative and semi-quantitative measurements of granulation tissue and inflammatory response were also assessed.

Quantitative analysis of clinical wound images showed that at days 7, 10, and 14, Collagen/ORC scaffolds containing 90 micrograms SCP lysate protein demonstrated statistically significant greater wound closure than the Collagen/ORC scaffold alone. In addition, at day 14, Collagen/ORC containing 30 micrograms cell lysate protein demonstrated statistically significant greater wound closure than Collagen/ORC (p<0.05, Tukey-Kramer for all).

Methods & Materials

A single 7.5 mm×7.5 mm full-thickness excisional wound was created on the left side of homozygous db/db mice and on heterozygous control mice. 56 mice were evaluated for 14 days.

The treatments were implanted at the time of surgery and left in place throughout the study period. The treatments (approx. 1×1 cm) were placed in the wound and covered with wound dressing pads sold under the tradename RELEASE (Johnson & Johnson, New Brunswick N.J.). The RELEASE pad was dipped into sterile saline and excess fluid was squeezed out prior to placing it on the animal. All wounds were then covered with transparent wound dressing sold under the tradename BIOCLUSIVE (Johnson & Johnson, New Brunswick N.J.).

Digital images of each wound were taken at days 0, 4, 7, 10, and 14 post-wounding. These images were used to evaluate wound closure over time.

Bandage changes were done on days 4, 7, and 10 of the study. Additional bandage changes were done if an animal escaped its bandage prior to a scheduled change.

Tissues were harvested from the animals on day 14. The entire wound and surrounding normal skin was excised and placed in 10% neutral buffered formalin. The cranial half of the excised tissue was sent for histological processing (paraffin sections) and stained with H&E and Masson's trichrome. The caudal portion of each sample was retained for possible future analysis.

Tissue sections were histologically analyzed for inflammatory response and quality of repair. Measurements of granulation tissue area and epithelial tongue length were also made.

Treatment Groups

Wound dressings, sold under the tradename PROMOGRAN (Johnson & Johnson, New Brunswick, N.J.), (Lot 1305263) was stored at room temperature prior to manipulation for this study. Cell lysate (CL) was aseptically applied to the scaffolds and then lyophilized under aseptic conditions. Scaffolds containing no CL were also lyophilized. The processed PROMOGRAN samples will be referred to as ORC/Collagen.

| Complete Description | As Referred to in Report |
|---|---|
| A. Saline treated (heterozygous control | db/db +/− |
| animal) | |
| B. Saline | Saline |
| C. Collagen/ORC | Collagen/ORC |
| D. Collagen/ORC + 30 ug cell lysate protein | Collagen/ORC + CL Low |
| E. Collagen/ORC + 90 ug cell lysate protein | Collagen/ORC + CL High |

N = 7 per treatment
The lot of cells used in treatments D & E were CBAT 120304.

Test Article Preparation

Lysate Production and Scaffold Preparation. Human hUTC lysate supernatant was prepared as in Example 22. The total protein content of the collected supernatant fluid was assessed by Bradford assay and the dose volume of supernatant fluid (30 micrograms total protein per material or 90 micrograms total protein per material) was calculated. The dose volume of supernatant fluid was applied to the material as five one-fifth total dose volume aliquots. An aliquot was placed at each corner of the 1.5×1.5 cm material approximately 1 mm from the material edge and one aliquot was placed in the center of the material. This ensured even distribution of lysate within the wound bed.

Lyophilization. Test materials with applied lysate were loaded into a FTS Systems Dura-Stop MP Stoppering Tray Dryer and lyophilized using the ramping program set forth in Example 17. All steps had a ramping rate of 2.5° C./minute and a 100-mT vacuum.

Anesthesia, Analgesia and Surgical Preparation. Each animal was weighed and tested for blood glucose level prior to anesthesia. Induction of anesthesia was accomplished by placing each mouse into a pre-charged Isoflurane anesthesia chamber. Once anesthetized, the animal was placed on a nose-cone to maintain the surgical plane of anesthesia. Eye ointment was applied to each animal to prevent corneal ulceration. No analgesics were administered due to the db/db mouse's physiology. Each animal was carefully scrutinized to determine if they were experiencing pain. Analgesics would have been administered if signs had been demonstrated.

Skin depilation from the back, shoulder, side and flank regions was accomplished with an electric animal clipper. The area was vacuumed to remove hair clippings and stratum corneum debris. Each animal was wiped with Betadine and alcohol prior to being placed on the surgical table.

Surgical Approach. Full-thickness excisional wounds (7.5×7.5 mm) were created on the left side of each animal with a scalpel and scissors. Each wound was submitted to a treatment regimen. The scaffolds were placed into the wound bed. CL treated scaffolds were placed “top-side” down.

Bandaging Technique. The test materials were undisturbed for the length of the study. The wounds were covered with an approximate 1×1 cm square of RELEASE. The RELEASE was dipped in sterile saline and the excess fluid was squeezed out prior to application. The wounds were further dressed with BIOCLUSIVE to keep the wounds moist and to keep the test articles and RELEASE in place.

The secondary bandages (RELEASE and BIOCLUSIVE) were changed on days 4, 7, and 10 of the study. Care was taken to ensure that the wound was not disturbed during the dressing changes. Additional bandage changes were performed if an animal escaped it bandages prior to a scheduled change.

Post-Operative Care and Clinical Observations

After recovering from surgery and general anesthesia, each mouse was observed for behavioral signs of discomfort or pain. No signs of discomfort or pain were observed. Animals were returned to their cage when fully conscious and ambulatory.

The health status of each mouse was determined by general appearance and attitude, food consumption, fecal and urinary excretion, the presence of abnormal discharges and bandage integrity. Each mouse was observed twice daily during the first 36 hours following surgery. Following recovery from surgery, the observations were reduced to once daily until the end of the study.

Evaluations. At each bandage change and at the end of the study, any unique findings were recorded.

Euthanasia. At the predetermined time point (7 and 14 days post-wounding), the animals were euthanized via carbon dioxide. The animals were observed to ensure that respiratory function had ceased and there was no palpable cardiac function.

Tissue Processing. Immediately following euthanasia, each wound along with the underlying fat and margin of surrounding skin was excised. The wound was bisected into cranial and caudal halves. The cranial half of the wound was fixed in 10% neutral buffered formalin, processed and embedded in paraffin. Samples were sectioned at 5 microns and stained for H&E and Masson's trichrome by MPI Research. The caudal half of the wound was fixed in 10% neutral buffer formalin and is reserved for any future analysis.

Photographic Documentation. Digital images were taken of individual wounds on days 0, 4, 7, 10, and 14 post-wounding. These images were used to measure wound closure. Using Image Pro 4.0 Image Analysis software, each image was calibrated using the ruler-label included in the photo. The wound was traced to determine the area that remained open. Day 0 images were used as baseline and the percentage remaining open was calculated based on the day being evaluated versus the area of that wound on day 0.

Histological Assessments. A computer-controlled motorized programmable slide scanning system was used in the process of image acquisition. Separate images of high magnification fields were acquired from a microscope. The images were tiled to preserve the integrity of the entire histological specimen. This allows accurate measurement of the entire tissue sample.

Images from the light microscope were captured into the computer memory via CCD camera and frame grabber board and subsequently analyzed using Image Pro 4.0 Image Analysis software.

Histological assessments were performed by a consulting pathologist. Tissue sections were histologically analyzed for the presence of the scaffold, granulation tissue quality and inflammatory response.

Statistical Analysis. Treatments were assigned in a blocked fashion. Visual assessments were analyzed using JMP 4.0.4 software. Shapiro-Wilk-W Test was performed prior to data analysis to determine normality. Nominal and Ordinal data was analyzed using Chi-Square. Continuous data was analyzed using One-way ANOVA. Tukey-Kramer or Student-Newman-Keuls (SNK) test for multiple comparisons was performed to determine differences between groups following One-way ANOVA. A value of p<0.05 was used as the level of significance.

Results

Surgery and anesthetic recovery were uneventful. All animals tolerated bandaging well.

Some differences between the diabetic groups were seen in blood glucose level, however all db/db mice were sufficiently diabetic during the course of the study.

Clinical Observations Day 14. On each day of bandage change and at the time of necropsy, each animal was evaluated. Any unique observations were noted. Table 25-1 summarizes the findings.

TABLE 25-1
Clinical Observation on Day 14

| | | | | | ORC/ | ORC/ | | | |
|---|---|---|---|---|---|---|---|---|---|
| | | | | | Collagen + | Collagen + | | ORC + | ORC + |
| Treatment | | db/db | | ORC/ | CL | CL | | CL | CL |
| Obs. | Day | +/− | Saline | Collagen | Low | High | ORC | Low | High |
| Wet | 4 | 2/7 | 7/7 | 4/7 | 0/7 | 0/7 | 0/7 | 1/7 | 0/7 |
| wounds | 7 | 3/7 | 7/7 | 5/7 | 0/7 | 0/7 | 4/7 | 5/7 | 6/7 |
| | 10 | 0/7 | 0/7 | 0/7 | 0/7 | 0/7 | 0/7 | 0/7 | 3/7 |
| | 14 | 0/7 | 0/7 | 0/7 | 0/7 | 0/7 | 0/7 | 1/7 | 3/7 |
| Treatment | 4 | N/A | N/A | 3/7 | 0/7 | 0/7 | 0/7 | 0/7 | 2/7 |
| visible | 7 | N/A | N/A | 6/7 | 7/7 | 6/7 | 7/7 | 7/7 | 7/7 |
| | 10 | N/A | N/A | 4/7 | 5/7 | 2/7 | 0/7 | 6/7 | 4/7 |
| | 14 | N/A | N/A | 4/7 | 5/7 | 3/7 | 5/7 | 5/7 | 5/7 |
| Escaped | 4 | 4/7 | 0/7 | 0/7 | 0/7 | 0/7 | 0/7 | 0/7 | 0/7 |
| Bandage | 7 | 2/7 | 0/7 | 0/7 | 0/7 | 0/7 | 0/7 | 0/7 | 0/7 |
| | 10 | 1/7 | 0/7 | 0/7 | 0/7 | 0/7 | 0/7 | 0/7 | 0/7 |
| | 14 | 3/7 | 0/7 | 0/7 | 0/7 | 0/7 | 0/7 | 0/7 | 0/7 |

TABLE 25-2
Percentage of Wound Closure

| | Average | | | | | SEM | | | |
|---|---|---|---|---|---|---|---|---|---|
| | | | | Day | Day | | | Day | Day |
| | Day 0 | Day 4 | Day 7 | 10 | 14 | Day 4 | Day 7 | 10 | 14 |
| db/db +/− | 100 | 84.2 | 51.94 | 25.61 | 3.65 | 17.17 | 9.18 | 11 | 2.49 |
| ORC/Collagen | 100 | 122.22 | 117.97 | 85.02 | 61.5 | 9.96 | 8.71 | 8.99 | 7.27 |
| ORC/Collagen + | 100 | 90.58 | 93.47 | 82.63 | 56.1 | 6.53 | 7.59 | 7.74 | 7.2 |
| CL Low | | | | | | | | | |
| ORC/Collagen + | 100 | 90.02 | 78.37 | 52.76 | 28.51 | 7.28 | 5.74 | 4.93 | 5.92 |
| CL High | | | | | | | | | |
| Saline | 100 | 99.25 | 101.57 | 77.21 | 51.23 | 7.33 | 10.47 | 3.4 | 6.53 |

Wound Closure (Day 14). Quantitative analysis of clinical wound images (Table 25-2) shows that at days 7, 10, and 14, Collagen/ORC scaffolds containing 90 micrograms cell lysate protein demonstrated statistically significant greater wound closure than the Collagen/ORC scaffold alone. In addition, at day 14, Collagen/ORC containing 30 micrograms cell lysate protein demonstrated statistically significant greater wound closure than Collagen/ORC (p<0.05, Tukey-Kramer for all).

For the ORC/Collagen treated groups at days 7, 10, and 14, db/db+/−demonstrated statistically significant greater wound closure than ORC/Collagen and ORC/Collagen+CL Low. At days 7, 10 and 14, ORC/Collagen+CL High demonstrated statistically significant greater wound closure than ORC/Collagen. In addition, at day 14, ORC/Collagen+CL Low demonstrated statistically significant greater wound closure than ORC/Collagen (p<0.05, Tukey-Kramer for all).

Qualitative Histopathogical Assessments

Scaffold Visibility. Most scaffolds were visible in the histological sections.

Presence of Adipose Tissue Near Wound Surface. Several wounds in the db/db mice had adipose tissue near the wound surface.

Subcutaneous Fat Necrosis. At day 14, the db/db+/−group demonstrated statistically less subcutaneous fat necrosis than all other groups. (p<0.05, Tukey-Kramer).

Inflammation in Superficial Wound Bed. At day 14, the Saline treated group demonstrated less inflammation in the superficial wound bed than all Collagen/ORC treated groups. (p<0.05, Tukey-Kramer).

Inflammation in Subcutaneous Fat. As expected, db/db+/−demonstrated less inflammation in SQ fat than all Collagen/ORC treated groups (p<0.05, Tukey-Kramer).

Granulation Tissue in Wound Bed. As expected, the db/db+/−group demonstrated statistically more granulation tissue in the wound bed than all other groups (p<0.05, Tukey-Kramer).

Summarized Qualitative Histology Data.

Results of qualitative histology assessment are provided in Table 25-3.

TABLE 25-3
Summary of qualitative histological scoring - 14 Days
Post Wounding

| | | | | Adipose | | | | Granulation |
|---|---|---|---|---|---|---|---|---|
| | | | | Tissue Near | | Inflammation | | Tissue in |
| | Animal | Treatment | Scaffold | Wound | SQ Fat | in Superficial | Inflammation | Wound |
| | No. | Code | Visible? | Surface? | Necrosis | Wound Bed | in SQ Fat | Bed |
| db/db +/− saline | 1 | A | CE | CE | CE | CE | CE | CE |
| control | 2 | A | N | N | 0 | 1 | 0 | 4 |
| | 3 | A | N | N | 0 | 1.5 | 1 | 4 |
| | 4 | A | CE | CE | CE | CE | CE | CE |
| | 5 | A | N | N | 0 | 1 | 0 | 4 |
| | 6 | A | N | N | 0 | 1 | 0 | 4 |
| | 7 | A | N | N | 0 | 1 | 1 | 4 |
| db/db | 8 | B | N | N | 2 | 1 | 2 | 3 with ares |
| saline | | | | | | | | of LQ |
| control | 9 | B | N | Y | 0.5 | 1 | 0.5 | 1 |
| | 10 | B | N | Y | 1.5 | 1 | 2 | 1 |
| | 11 | B | N | Y - minor | 2 | 1 | 2 | 2 with |
| | | | | | | | | areas of LQ |
| | 12 | B | N | Y | 1 | Empty WB | 1 | 0.5 |
| | 13 | B | N | N | 1 | 1 | 1 | 1.5 LQ |
| | 14 | B | N | N | 1 | 1 | 1 | 1 LQ |
| Collagen/ | 15 | C | S | Y | 2 | 2 | 2 | 1 to 2.5 |
| ORC | | | | | | | | with areas |
| | | | | | | | | of rel-lq |
| | 16 | C | S | Y | 2 | 1.5 | 2 | 1.5 rel-LQ |
| | 17 | C | N | Y | 2 | 2 | 2 | 1.5 |
| | 18 | C | S | Y | 0.5 | 2 | 1 | 1.5 rel-LQ |
| | 19 | C | S | Y - minor | 0.5 | 1 | 1 | 1.5 rel-LQ |
| | 20 | C | S | Y | 1.5 | 2.5 | 2 | 1 |
| | 21 | C | N | Y | 1 | 2 | 2 | 1.5 LQ |
| Collagen/ | 22 | D | N | N | 1 | 1 | 1 | 1.5 rel-LQ |
| ORC + 30 ug | 23 | D | S | N | 0 | 1 | 0.5 | 1 LQ |
| lysate | 24 | D | S | Y | 2.5 | 3 | 2.5 | 2 with |
| | | | | | | | | areas LQ |
| | 25 | D | S | Y | 2 | 2 | 2 | 1 lq |
| | 26 | D | S | Y | 2 | 2 | 2 | 1.5 LQ |
| | 27 | D | S | Y | 2 | 2 | 2 | 1.5 LQ |
| | 28 | D | S | Y | 2.5 | 2 | 2 | 1 LQ |
| Collagen/ | 29 | E | S | Y - rel minor | 2.5 | 2.5 | 2.5 | 2 with |
| ORC + 90 ug | | | | | | | | areas LQ |
| lysate | 30 | E | S | Y | 2.5 | 2 | 2.5 | 1.5 with |
| | | | | | | | | areas LQ |
| | 31 | E | Partial S | Y - rel minor | 1 | 1 | 1 | 1 LQ |
| | 32 | E | S | Y - rel minor | 1 - PF | 2 | 2 - PF | 3* |
| | 33 | E | S | N | 1.5 - PF | 2 | 1.5 - PF | 1 LQ |
| | 34 | E | S | N | 1.5 - PF | 2 | 1.5 - PF | 1.5 mainly |
| | | | | | | | | LQ |
| | 35 | E | N | Y | 2.5 | 2 | 2.5 | 1.5 mainly |
| | | | | | | | | LQ |

Table Key
CE = cannot evaluate,
S = sloughing,
N = no,
NN = not notable (NN = 0 for mean calculations)

Summary. The purpose of this study was to evaluate the biological effect of hUTC lysate lyophilized onto and released from a natural scaffold consisting of collagen/ORC in a recognized model of delayed healing, the db/db mouse wound healing model. The primary endpoint considered in this evaluation was the effect on the increase in the healing rate (time to complete wound closure) in this impaired model since this is the key requirement set forth from the FDA Guidance for Industry for Development of products for treatment in cutaneous ulcers.

Quantitative analysis of clinical wound images shows that at days 7, 10 and 14, Collagen/ORC scaffolds containing 90 microgram cell lysate protein demonstrated statistically significant greater wound closure than the Collagen/ORC scaffold alone. In addition, at day 14, Collagen/ORC containing 30 microgram cell lysate protein demonstrated statistically significant greater wound closure than Collagen/ORC (p<0.05, Tukey-Kramer for all).

These results demonstrate the ability of hUTC lysate, lyophilized onto and released from a natural biomaterial of collagen/ORC, to increase the rate of closure in a db/db mouse full thickness wound healing model.

While the present invention has been particularly shown and described with reference to the presently preferred embodiments, it is understood that the invention is not limited to the embodiments specifically disclosed and exemplified herein. Numerous changes and modifications may be made to the preferred embodiment of the invention, and such changes and modifications may be made without departing from the scope and spirit of the invention as set forth in the appended claims.

What is claimed:

1. A composition comprising at least one stem cell product having the potential to provide support to a cell.

2. The composition of claim 1 wherein the stem cell products are derived from cells selected from the group consisting of blastocysts, trophoblasts, the inner cells mass, embryonic germ cells, placenta, umbilical cord, amnioic epithelium, amnionic membrane, amnionic fluid, mesenchymal stem cells, adipose derived stem cells, epidermal derived stem cells, hair follicle derived stem cells, mammary tissue derived stem cells, olfactory derived stem cells, neural stem cells, epithelial stem cell, cardiac derived stem cells, stem cells derived from teeth, and hematopoietic stem cells.

3. The composition of claim 1 comprising one or more bioactive factors.

4. The composition of claim 3 wherein said bioactive factor is at least one of a differentiation-inducing factor, an anti-apoptotic agent, an anti-inflammatory agent, an immunosupressive/immunomodulatory agent, an anti-proliferative agent, a corticosteroid, an antibody, an anti-thrombogenic agent, an anti-oxidant, and scar inhibitory factor.

5. A pharmaceutical composition comprising the stem cell product of claim 1 and a pharmaceutically acceptable carrier.

6. The composition of claim 1, wherein the stem cell product is selected from the group consisting of a soluble cell fraction, an insoluble cell fraction, a cell membrane-containing fraction, a cell cytoplasm-containing fraction, a cell nucleus-containing fraction, a cell lysate, a supernatant of cell fraction, a conditioned medium, an extracellular matrix; a trophic factor and combinations thereof.

7. A method of providing trophic support to a soft tissue cell by exposing said soft tissue cell to the stem cell product of claim 1.

8. A matrix comprising the stem cell product of claim 1.

9. A method of treating a soft tissue condition in a patient comprising administering to said patient a therapeutically effective amount of the stem cell product of claim 1.

10. A kit comprising at least one stem cell product of claim 1 and at least one additional component selected from the group consisting of a matrix, a hydrating agent, a cell culture substrate, a bioactive factor, a cell type, a differentiation-inducing agent, and cell culture media.

11. The kit of claim 10 additionally comprising instructions for use thereof.
